(19) United States
(12) Patent Application Publication
(10) Pub. No.: US 2021/0369583 A1
(43) Pub. Date: Dec. 2, 2021

(54) COMPOSITIONS FOR PROVIDING SKIN CARE BENEFITS AND METHODS OF USE

(71) Applicant: Danisco US Inc., Palo Alto, CA (US)

(72) Inventors: Carole GHERADI, Savigny (FR); Andreas J. KROUWER, Leiden (NL); Piera Mattia PERICU, Mannheim (DE); Heli PUTALAA, Kirkkonummi (FI); Vincent A.L. WORTEL, Leiden (NL)

(21) Appl. No.: 16/971,509

(22) PCT Filed: Feb. 7, 2019
(86) PCT No.: PCT/US2019/016982

§ 371 Date: Aug. 20, 2020

Related U.S. Application Data
(60) Provisional application No. 62/741,755, filed on Oct. 5, 2018.
(60) Provisional application No. 62/645,236, filed on Mar. 20, 2018.
(60) Provisional application No. 62/635,157, filed on Feb. 26, 2018.

(51) Int. Cl. A61K 8/60; A61K 8/49; A61K 8/44; A61Q 19/00; A61Q 17/04; A61K 8/02; A61K 8/06; A61K 8/04

(52) U.S. Cl. CPC A61K 8/60; A61K 8/4913; A61K 8/44; A61Q 19/00; A61Q 19/007; A61Q 17/04; A61Q 17/00; A61K 8/0229; A61K 8/062; A61K 8/042; A61K 2800/524; A61K 2800/262; A61K 8/0212

(57) ABSTRACT

The present disclosure is directed to compositions and methods for providing increased skin care benefits and/or multiple skin care benefits. The methods and compositions disclosed herein are directed to skin care compositions and skin care products comprising at least four osmolytes selected from amino acids, methyl-amines and polyols. More specifically, the methods and compositions disclosed herein are directed to skin care compositions and skin care products comprising a combination of at least four osmolytes selected from the group consisting of betaine, inositol, proline and serine.

This application claims the benefit of U.S. Provisional Application No. 62/635,157, filed Feb. 26, 2018, U.S. Provisional Application No. 62/645,236, filed Mar. 20, 2018 and U.S. Provisional Application No. 62/741,755, filed Oct. 5, 2018, each of which incorporated by reference herein in their entireties.

### FIELD OF THE DISCLOSURE

The present disclosure is directed towards skin care compositions, skin care products, and methods for providing increased skin care benefits and/or multiple skin care benefits. More specifically, the present disclosure is directed towards methods and compositions comprising a combination of at least the osmolytes betaine, inositol, proline and serine.

### BACKGROUND

The skin functions as a barrier protecting the organism from drying out as well as protecting the organism against the penetration of external, often harmful, substances.

The epidermis constitutes the outermost region of the skin tissue and as such forms the actual protective sheath against the environment.

The outer layer of the epidermis (Stratum corneum or Horny layer) is the part which is in contact with the environment and the particular structure of the horny layer protects the skin as well as stabilizes its own flexibility by binding a defined amount of water (P. M. Elias, Structure and Function of the Stratum Corneum Permeability Barrier, Drug Dev. Res. 13, 1988, 97-105).

Osmolytes are compounds that are accumulated by the cell in response to a hyperosmotic stress such as dehydration, and which are then rapidly released therefrom (Kwon and Handler, Current Opin. Cell. Biol.; 1995; 7: 465-471 and Haussinger 1996; Biochem. J. 313: 697-710). Osmolytes perturb the cell very little even at high concentrations and, furthermore, do not interfere with the functions of the proteins of said cells (Burg et al., Annu. Rev. Physiol. 1997; 59: 437-455).

There remains a need to find skin care compositions and skin care products that provide an increased skin care benefit and/or multiple skin care benefits such as but not limiting to increased protection against the loss of substances endogenous to the body (e.g. water, lipids, electrolytes) as well as an increased protection against harmful external chemical and physical influences, or any combinations thereof.

### SUMMARY

The present disclosure is directed to compositions and methods for providing increased skin care benefits. The present disclosure is also directed to compositions and methods for providing multiple skin care benefits. The methods and compositions disclosed herein are directed to skin care compositions and skin care products comprising at least four osmolytes selected from amino acids, methyl-amines and polyols. More specifically, the methods and compositions disclosed herein are directed to skin care compositions and skin care products comprising a combination of at least four osmolytes selected from the group consisting of betaine, inositol, proline and serine.

In one embodiment, the skin care composition is a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition.

In one embodiment, the skin care composition is a skin care composition comprising a combination of at least four osmolytes selected from the group consisting of proline, serine, betaine, inositol, wherein said combination comprises 30% proline, 14% serine, 50% betaine and 6% inositol on a % osmolyte basis relative to a total osmolyte content of 100%.

The skin care compositions described above can further contain at least one additional compound selected from the group consisting of preservatives, pH adjusters, anti-oxidants and chelators.

In one embodiment the use is a use of an effective amount of the skin care composition described herein in a skin care product.

In one embodiment, the skin care product is a skin care product comprising an effective amount of the skin care composition described herein and one or more dermatologically or cosmetically acceptable components. In one aspect, the effective amount of the skin care composition described herein can be at least about 0.1% to 5% on a weight basis relative to a total weight of said skin care product. In one aspect, the effective amount of the skin care composition described herein can be at least about 0.1% to 10% on a weight basis relative to a total weight of said skin care product. The dermatologically acceptable component can be a dermatologically acceptable carrier comprising about 10% to about 99% on a weight basis relative to a total weight of the skin care product.

In one embodiment, the skin care product is a lotion, a serum, a jelly, a cream, a gel, an emulsion, a mask, a patch, or a stick comprising one or more dermatologically or cosmetically acceptable components and at least about 1%, 2%, 3%, 4%, 5%, 6%, 7%, 8%, 9% up to 10% of the skin care composition described herein on a weight basis relative to a total weight of said skin care product.

In one embodiment, the skin care product is a skin care product comprising a combination of the osmolytes proline, serine, betaine and inositol in a ratio of 30:14:50:6.

In one embodiment, the method is a method for providing an increased skin care benefit to skin (including skin surface, skin cell), said method comprising contacting a skin with a skin care product comprising an effective amount of a skin care composition described herein and one or more dermatologically or cosmetically acceptable components. The increased skin care benefit can be selected from the group consisting of an increased osmoprotection, an increased UV irradiation protection, and a combination thereof.

In one embodiment, the method is a method for providing an increased skin care benefit to a skin, said method comprising contacting a skin with a skin care product comprising a combination of at least four osmolytes selected from the group consisting of betaine, inositol, proline and serine, wherein said combination comprises 1% to 50% on a weight basis relative to a total weight of said skin care product.

In one embodiment, the method is a method for providing multiple skin care benefits to a skin, said method comprising contacting a skin with a skin care product comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, wherein said multiple skin care benefits are at least two skin care benefits selected from the group consisting of skin moisturizing, osmoprotection of the skin, protection of the skin against pollution, protecting of the skin against ultra violet irradiation damage, protecting of the skin against blue light damage, protecting of the skin against oxidative stress, skin detoxification, strengthening the skin barrier function, and any one combination thereof.

Skin care benefits include, but are not limited to, osmoprotection, UV irradiation protection, blue light protection, night light protection, protection against oxidative stress, skin detoxification, improved skin barrier function, or any one combination thereof.

### DETAILED DESCRIPTION

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination.

### Skin Care Compositions

As used herein the term “skin care composition” refers to a composition comprising at least one skin care benefit agent capable of providing a skin care benefit.

As used herein the term “skin care benefit agent” refers to a compound that can provide a skin care benefit.

As used herein the term “skin care benefit” refers to a benefit provided by the skin care composition, or skin care product comprising an effective amount of said skin care composition when applied to a skin, wherein the benefit is selected from the group consisting of skin moisturizing (protecting the skin against dehydration by maintaining, restoring and/or strengthening the moisturization of the skin), osmoprotection of the skin (protecting the skin against the loss of substances endogenous to the body, e.g. water, lipids, electrolytes), protecting the skin against environmental stresses (such as chemicals, microorganisms, allergens), protecting the skin against pollution, protecting the skin against ultra violet irradiation damage, protecting the skin against blue light, protection against oxidative stress, skin detoxification, preventing or retarding the appearance of the signs of aging of the skin, reducing the appearance of skin wrinkles, skin rejuvenation, reducing dermal adhesions, promoting cell or tissue longevity, strengthening the skin barrier function, or any one combination thereof.

As used herein, skin care benefit agents include, but are not limited to, osmolytes selected from amino acids, methyl-amines and polyols. More specifically, skin care benefit agents include the osmolytes betaine, inositol, proline, serine, or any functional equivalent or derivative thereof.

Betaine exists in plants, like sugar beet, and can be recovered as a natural product from sugar beet extracts and side streams of sugar production (such as sugar beet molasses, rest molasses, betaine containing fractions, betaine molasses, mother liquor from betaine crystallization or vinasse) or fermentation (U.S. Pat. Nos. 5,127,957, 6,093,326, WO2007080228 published Jul. 19, 2007). Betaine is an amphoteric compound and is water soluble.

Betaine is a natural sugar beet extract that has a moisturization and osmolytic effect and can serve as a methionine choline replacement (Warskulat U. J Invest Dermatol 123:516-521, 2004).

In one aspect, the betaine used in the present disclosure may be any natural betaine, including but not limiting to, natural glycine betaine (natural trimethylglycine), natural histidine betaine, natural proline betaine and natural tyrosine betaine.

In an aspect of the present disclosure, a derivative of the betaine may be used. For example, an organic acid salt, an inorganic acid, a base salt, etc. may be used.

As used herein the term “inositol” includes 1,2,3,4,5,6-hexahydroxycyclohexane and myo-inositol. Inositol exists in plants, like sugar beet, and can be recovered as a natural product from sugar beet extracts, sugar beet molasses and side streams of sugar production or fermentation (WO2007/080228 published Jul. 19, 2007; U.S. Pat. No. 6,093,326).

Myo-inositol exists in nature either in its free form (found, for example, in sugarcane, beet molasses, and almond hulls) or as a hexaphosphate called phytin (found, for example, in corn steep liquor) (Warskulat U. J Invest Dermatol 123:516-521, 2004). Inositol and inositol hexaphosphate can also be obtained from rice.

As used herein the term “Proline” includes L-Proline and D-Proline.

Proline, an amino acid, plays a beneficial role in plants exposed to various stress conditions. Besides acting as an excellent osmolyte, proline can act as a metal chelator, an anti-oxidative defense molecule, and a signaling molecule when an organism is exposed to stress. (Shamsul Hayat., Plant Signaling & Behaviour 7:11, 1456-1466, November 2012).

As used herein the term “Serine” includes L-Serine and D-Serine.

Serine, an amino acid, can act as a neutral osmolyte (Arakawa T, et. al. Biophys J 1985 March, 47 (3):411-4).

In one aspect, the skin care composition comprises a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition.

In one aspect, the skin care composition comprises 15% Proline, 7% serine, 25% betaine, 3% inositol and on a weight basis relative to a total weight of said composition.

In one aspect the skin care composition comprises the osmolytes proline, serine, betaine, inositol in a ratio of 30:14:50:6 to total osmolytes, respectively.

The skin care composition can comprise additional compounds selected from the group consisting of preservatives, pH adjusters, anti-oxidants and chelators.

Preservatives include but are not limited to parabens, sodium benzoate, potassium sorbate, phenyl ethyl alcohol, Lauryl ethyl arginate (LAE) and any combination thereof.

pH adjusters include but are not limited to weak acids, strong acids, any compound that can adjust the pH, such as but not limiting to citric acid, or any combination thereof.

In one aspect the skin care composition further comprises the preservative sodium benzoate at 0.4 wt % and/or pH adjuster citric acid at 0.8 wt %

Antioxidants include, but are not limited to, BHA, BHT and tocopherol.

The skin care composition described herein can be an aqueous composition. The skin care composition can have a pH of about 3-12. In one aspect the skin care composition has a pH of about 4.0 to 5.0. The skin care composition described herein can be used in a skin care product, wherein the pH of the skin care product can be further adjusted as desired for said skin care product.

The skin care composition described herein can be a colorless, clear, homogeneous composition without having any precipitate or crystal or fibers present.

An effective amount of the skin care compositions described herein can be used in formulations and skin care products.

An effective amount of the skin care composition in a formulation or skin care product can be the amount of the skin care composition described herein that helps provide a reduction in the harmful effects of xenobiotics (pollutants), and empowers the skin natural response to fight against environmental stresses. For example, an effective amount of the skin care composition in a formulation or skin care product can be the amount that provides pollution protection and osmoprotection benefits, by contributing to protect the skin via three main defence strategies: anti-oxidation, detoxification and skin barrier function improvement, and helps the skin to maintain its natural moisture balance.

As described herein, an effective amount of the skin care composition in a formulation or skin care product can be the amount that helps protect skin cells, skin surface or skin from oxidative stress caused by urban pollution and UV, as is evidenced by a reduction of pollution induced lipids peroxidation (MDA test described herein), a reduction of UV-induced protein carbonylation (FTZ marker test described herein) and a reduction of UV-induced DNA damage (TUNEL marker test described herein).

As described herein, an effective amount of the skin care composition in a formulation or skin care product can be the amount that contributes to skin (skin cells, skin surface) detoxification, as is evidenced by an induction of oxidative stress transcription factors (Aryl Hydrocarbon Receptor (AhR) test and Nuclear factor erythroid 2-related factor (Nrf2) test described herein), an induction of anti-oxidant enzyme (Heme-Oxygenase 1 (HO-1) test described herein) and a contribution to limit the effects of heavy metal exposure as indicated by a reduction of the pollution incudes activation Metallothionein 1 (MT-1H) protein.

As described herein, an effective amount of the skin care composition in a formulation or skin care product can be the amount that improves the skin barrier function by contributing to strengthening keratinocytes tight junctions (Trans Epithelial Electrical Resistance (TEER) test described herein), and a decreased substance diffusion (Caffeine diffusion test described herein).

In one embodiment, the skin care composition is a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, wherein an effective amount of said composition in a formulation or skin care product provides a skin care benefit selected from the group consisting of a pollution protection (such as but not limited to protection from night pollution), an osmoprotection, or any one combination thereof.

Pollution is often at its peak between the early evening hours and midnight, (referred herein as night pollution) for example due to fine dust settling from the air. In one aspect, an effective amount of the skin care composition described herein can be the amount of the skin care composition described herein that helps provide pollution protection, such as night pollution protection, by reinforcing the skin barrier, protecting against oxidative stress and detoxifying the skin.

In one embodiment, the skin care composition is a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, wherein an effective amount of said composition in a formulation or skin care product provides a skin care benefit selected from the group consisting of a decrease in oxidative stress, an increased detoxification, and increased skin barrier function, or any one combination thereof.

In one embodiment, the skin care composition is a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, wherein an effective amount of said composition in a formulation or skin care product provides a skin care benefit selected from the group consisting of a reduction of lipid peroxidation, a reduction of UV-induced protein carbonylation, a reduction of UV-induced DNA damage, a reduction of ROS under UV exposure, an increase in oxidative stress transcription factors, an increase in anti-oxidant enzymes, a reduction of induced activation of MT-1H, a strengthening of the keratinocyte tight junctions, a decrease of caffeine diffusion, or any one combination thereof.

In one embodiment, the skin care composition is a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, wherein the effective amount of said skin care composition is at least about 0.1% to 5% on a weight basis relative to a total weight of said formulation or skin care product.

In one aspect, the effective amount of the skin care composition in a formulation or skin care product can be at least about 0.3% to 5.0%, on a weight basis relative to a total weight of said formulation or skin care product. The range is intended to include the endpoints thereof, and all integers and fractions within the range.

In one aspect, the effective amount of the skin care composition described herein can be at least about 0.1% to 10% on a weight basis relative to a total weight of said skin care product. An effective amount of the skin care composition in a formulation or skin care product includes at least 0.1%, 0.2%, 0.3%, 0.4%,0.5%, 0.6%, 0.7%, 0.8%, 0.9%, 1.0%, 1.1%, 1.2%, 1.3%, 1.3%, 1.4%,1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, to 10% skin care composition on a weight basis relative to a total weight of said formulation or skin care product.

### Skin Care Products

The skin care compositions or any effective amount of said skin care composition described herein can be used in skin care products.

As used herein “skin care products” refer to products comprising an effective amount of the skin care compositions described herein, including but not limiting to aqueous solutions, emulsions, serums, jellies, masks, patches, face masks, peel-off masks, lotions, topical moisturizers, creams, pastes, balms, ointments, pomades, gels, liquids, sprays, foam, kits, or any one combinations thereof.

A skin care product, can also be in the form of makeup, lipstick, mascara, foundation, blush, eyeliner, lip gloss, macro emulsion, other cosmetics, sunscreen, sun block, bath gel, shower gel, body wash, face wash, skin conditioner, cold cream, moisturizer, body spray, soap, body scrub, for example.

A skin care product includes a liquid lotion (true solution) comprising water as a solvent and water soluble additives (solutes), such as but not limiting to an active, a fragrance, a color, a preservative, a pH adjuster, a chelating agent, or any one combination thereof.

A skin care product includes a dispersion such as an emulsion (such as, but not limited to the following: liquid in liquid [water in oil W/O, O/W, W/O/W], suspension [solid/liquid or liquid/solid], aerosol [liquid/gas or solid/gas], foam/mousse [gas/liquid or gas/emulsion, or gas/solid]). An example of an Oil in Water [O/W] emulsion includes, but is not limited to a combination of a water phase, an emulsifier, a fatty phase and an at least one additive. The water phase can comprise water, humectants and stabilizing agents [such as, but not limiting to, synthetic polymers, carbomers, natural polymers, xanthan gum, acacia gum, carragheenan, gellan, or any one combination thereof). Emulsifiers include, but are not limited to, anionic emulsifiers, cationic emulsifiers, non-ionic emulsifiers, amphoteric emulsifiers, silicone emulsifiers), autoemulsifying agents. Fatty phases (lipophilic ingredients) include, but are not limited to, waxes, butter, fatty esters, triglycerides, vegetal oil, mineral oil (parffinum), silicones, and thickeners/oil jellifying agents. Additives include, but are not limited to, preservative, fragrance (most often lipophilic), color, anti-oxidant, chelating agent, actives, pH adjuster (citric acid, lactic acid, AHA), neutralizers/strong basic agent like NaOH, Trimethylamine (for acrylic polymers to jellify) and powders.

A skin care product includes an aqueous gel comprising a water phase (including water, humectants, actives), a jellifying agent (such as but not limited to synthetic polymers, natural polymers, xanthan gum, acacia gum, carragheenan, gellan) and an additive (such as but not limited to fragrance, high HLB surfactant, color, actives, preservative system, pH adjuster, neutralizing agent, powders).

A skin care product includes a cleansing/surfactant system (such as but not limited to a shampoo, shower gel, micellar water) comprising a water phase (water, humectants), a surfactant, an additive (such as but not limited to fragrance, high HLB surfactant, color, actives, preservative system, pH adjuster, neutralizing agent, powders) and optionally a jellifying agent (such as but not limited to synthetic polymers, natural polymers, xanthan gum, acacia gum, carragheenan, gellan).

As described herein, a skin care product or formulation comprising an effective amount of the skin care composition comprising the osmolytes of proline, serine, betaine and inositol as described herein, provides a skin care benefit selected from the group consisting of a decrease in oxidative stress, an increased detoxification, and increased skin barrier function, or any one combination thereof.

As described herein, a skin care product or formulation comprising an effective amount of the skin care composition comprising the osmolytes of proline, serine, betaine and inositol as described herein, provides a skin care benefit selected from the group consisting of a reduction of lipid peroxidation, a reduction of UV-induced protein carbonylation, a reduction of UV-induced DNA damage, a reduction of ROS under UV exposure, an increase in oxidative stress transcription factors, an increase in anti-oxidant enzymes, a reduction of induced activation of MT-1H, a strengthening of the keratinocyte tight junctions, a decrease of caffeine diffusion, or any one combination thereof. The effective amount of the skin care composition in the above listed skin care product or formulation can be at least about 0.1% to 5% on a weight basis relative to a total weight of said formulation or skin care product.

In one embodiment, the skin care product is a skin care product comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, wherein said effective amount of said skin care composition provides a skin care benefit selected from the group consisting of a decrease in oxidative stress, an increased detoxification, and increased skin barrier function, or any one combination thereof. The effective amount of the skin care composition in said skin care product can be at least about 0.1% to 5%, at least about 0.1% to 6%, at least about 0.1% to 7%, at least about 0.1% to 8%, at least about 0.1% to 9%, at least about 0.1% to 10% on a weight basis relative to a total weight of said formulation or skin care product. In one aspect the effective amount of the skin care composition in said skin care product can be at least about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, to 10% on a weight basis relative to a total weight of said formulation or skin care product.

In one embodiment, the skin care product is a skin care product comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, wherein said effective amount of said skin care composition provides a skin care benefit selected from the group consisting of a reduction of lipid peroxidation, a reduction of UV-induced protein carbonylation, a reduction of UV-induced DNA damage, a reduction of ROS under UV exposure, an increase in oxidative stress transcription factors, an increase in anti-oxidant enzymes, a reduction of induced activation of MT-1H, a strengthening of the keratinocyte tight junctions, a decrease of caffeine diffusion, or any one combination thereof. The effective amount of the skin care composition in said skin care product can be at least about 0.1% to 5% on a weight basis relative to a total weight of said formulation or skin care product.

As described herein, it has been found surprisingly and unexpectedly that when the osmolytes of proline, serine, betaine and inositol are combined in a skin care composition or skin care product an increased skin care benefit was obtained.

In one aspect, the increased skin care benefit can be evidenced by an increased osmoprotection, thereby enabling to restore, maintain or strengthen the moisturization of the skin.

As used herein the term “increased skin care benefit” includes a benefit obtained from applying a skin care composition (or a skin care product comprising an effective amount of said composition) to a skin, wherein said benefit is greater than the skin care benefits of a reference composition (reference skin care product) comprising an equal amount of total osmolytes compared to the skin care composition (skin care product).

For example, an increased skin care benefit is observed when the skin care benefit of a skin care composition (or skin care product) comprising W mg/ml betaine, X mg/ml inositol, Y mg/ml proline and Z mg/ml serine, wherein the total osmolyte concentration is T (T=W+X+Y+Z mg/ml) is greater than the skin care benefit obtained from a reference composition (or reference product) comprising only one osmolyte (selected from either betaine, inositol, proline or serine) at a total concentration of T mg/ml.

For example, as described herein, in one aspect, a skin care product can comprise a combination of osmolytes in an amount of 3 mg/ml L-proline, 1.4 mg/ml L-serine, 5 mg/ml serine and 0.6 mg/ml inositol totaling 10 mg/ml of osmolytes. The skin care benefit obtained from the above skin care product can be compared to the skin care benefit obtained from a reference product comprising only one osmolyte (selected from betaine, inositol, proline or serine) at a total concentration equal to the total osmolyte concentration of the above the skin care composition, e. g 10 mg/ml proline. If the skin care benefit obtained from the skin care product is greater than the skin care benefit from the reference product, the skin care product provides an increased skin care benefit.

As used herein the term “increased skin care benefit” also includes a benefit obtained from applying a skin care composition (or a skin care product comprising an effective amount of said skin care composition) comprising a combination of at least four osmolytes to a skin, wherein said benefit is greater than sum of all benefits obtained from each individual component of said combination, thereby providing said increased skin care benefit. A such, an increased skin care benefit is observed when the skin care benefit of a skin care composition (or skin care product) comprising W mg/ml betaine, X mg/ml inositol, Y mg/ml proline and Z mg/ml serine, wherein the total osmolyte concentration is T (T=W+X+Y+Z mg/ml) is greater than the sum of [the skin care benefit of a reference composition (reference product) comprising W mg/ml betaine as the sole osmolyte+the skin care benefit of a reference composition (reference product) comprising, X mg/ml inositol, the skin care benefit of a reference composition (reference product) comprising Y mg/ml proline as the sole osmolyte+the skin care benefit of a reference composition (reference product) comprising Z mg/ml serine as the sole osmolyte], thereby providing said increased skin care benefit.

As used herein the term “increased skin care benefit” also includes a benefit obtained from applying a skin care composition (or a skin care product comprising an effective amount of said composition) comprising a combination of at least four osmolytes to a skin, wherein said benefit is greater than sum of all benefits obtained from each individual component of said combination, thereby providing said increased skin care benefit. A such, an increased skin care benefit is observed when the skin care benefit of a skin care composition (skin care product) comprising w % betaine, x % inositol, y % proline and z % serine (on a weight % basis) is greater than the sum of [the skin care benefit of a reference composition (reference product) comprising w % betaine as the sole osmolyte+the skin care benefit of a reference composition (reference product) comprising x % inositol, the skin care benefit of a reference composition (reference product) comprising y % proline as the sole osmolyte+the skin care benefit of a reference composition (reference product) comprising z % serine as the sole osmolyte], thereby providing said increased skin care benefit.

As described herein, it has been found surprisingly and unexpectedly that when the osmolytes of proline, serine, betaine and inositol are combined in a skin care composition described herein or skin care product comprising an effective amount of said skin care composition, an increased osmoprotection of cells exposed to a hyperosmotic stress was obtained.

As described herein, skin care products comprising a combination of proline, serine, betaine and inositol were capable of helping protect skin against oxidative stress as evidenced by a reduced pollution induced lipids peroxidation (MDA marker), reduced UV-induced carbonylated proteins (FTZ marker), and reduced UV-induced DNA damage (TUNEL marker).

Furthermore, as described herein, skin care compositions and skin care products comprising a combination of proline, serine, betaine and inositol contributed to skin detoxification, as evidenced by induced oxidative stress transcription factors AhR (Aryl Hydrocarbon Receptor) and Nrf2 (Nuclear factor erythroid 2-related factor) and anti oxidant enzyme HO-1 (Heme-oxygenase 1), and also by decreased metal regulation protein MT-1H (Metallothionein 1) induced by pollution.

Furthermore, as described herein, skin care compositions and skin care products comprising a combination of proline, serine, betaine and inositol were able to improve the skin's barrier function, as was evidenced by strengthened keratinocytes tight junctions (TEER test) and decreased substances diffusion (Caffeine diffusion test).

As described herein, skin care compositions and skin care products comprising a combination of proline, serine, betaine and inositol were capable of providing skin protection against pollution.

Skin care products described herein may further comprise one or more dermatologically or cosmetically acceptable components known or otherwise effective for use skin care, provided that the optional components are physically and chemically compatible with the essential components described herein, or do not otherwise unduly impair product stability, aesthetics, or performance. Non-limiting examples of such optional components are disclosed in International Cosmetic Ingredient Dictionary, Ninth Edition, 2002, and CTFA Cosmetic Ingredient Handbook, Tenth Edition, 2004.

In one aspect, the dermatologically or cosmetically acceptable component is a dermatologically acceptable carrier comprising from about 10 wt. % to about 99.9 wt. %, alternatively from about 50 wt. % to about 95 wt. %, and alternatively from about 75 wt. % to about 95 wt. %, of a dermatologically acceptable carrier. Carriers suitable for use with the composition(s) may include, for example, those used in the formulation of mousses, tonics, gels, skin moisturizers and lotions. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one aspect, the carrier comprises water, fatty alcohols, volatile organic alcohols, and mixtures thereof. Other carriers can be formulated by those of ordinary skill in the art.

The skin care products described herein may further comprise from about 0.1 to about 10%, and alternatively from about 0.2% to about 5.0%, of a gelling agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked alkyl ether/acrylate copolymers (e.g., SALCARE™ SC90 commercially available from Allied Colloids); unneutralized crosslinked copolymers of sodium polyacrylate, mineral oil, and PEG-1 trideceth-6 (e.g., SALCARE™ SC91 commercially available from Allied Colloids); unneutralized crosslinked copolymers of methyl vinyl ether and maleic anhydride (e.g., STABILEZE™ QM-PVM/MA copolymer commercially available from International Specialty Products); hydrophobically modified nonionic cellulose polymers; hydrophobically modified ethoxylate urethane polymers (e.g., UCARE™ Polyphobe Series of alkali swellable polymers commercially available from Union Carbide); and combinations thereof. In this context, the term “unneutralized” means that the optional polymer and copolymer gelling agent materials contain unneutralized acid monomers.

The cosmetically acceptable medium may contain a fatty substance in a proportion generally of from about 10 to about 90% by weight relative to the total weight of the product, where the fatty phase containing at least one liquid, solid or semi-solid fatty substance. The fatty substance includes, but is not limited to, oils, waxes, gums, and so-called pasty fatty substances. Alternatively, the products may be in the form of a stable dispersion such as a water-in-oil or oil-in-water emulsion. Additionally, the skin care products may contain one or more conventional cosmetic or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments (colorant agents).

The dermatologically acceptable carrier may be a moisturizer formulation containing at least one emulsifiers, at least one surfactant, or any combination thereof.

Skin care products can further comprise skin care active ingredient materials including sun screen agents, moisturizers, humectants, benefiting agents skin, depositing agents such as surfactants, occlusive agents, moisture barriers, lubricants, emollients, anti-aging agents, antistatic agents, abrasive, antimicrobials, conditioners, exfoliants, fragrances, viscosifying agents, salts, lipids, phospholipids, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons, and mixtures thereof.

Other ingredients that may be included in a skin care product include, without limitation, at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these, one or more natural moisturizing factors (such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example), glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

In one embodiment the skin care product is a skin care product comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more dermatologically or cosmetically acceptable components.

In one embodiment the skin care product is a skin care product comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more dermatologically or cosmetically acceptable components, wherein said effective amount of the skin care composition is at least about 1%, 2%, 3%, 4% up to 5% on a weight basis relative to a total weight of said skin care product.

In one embodiment the skin care product is a skin care product comprising a combination of the osmolytes proline, serine, betaine and inositol in a ratio of 30:14:50:6.

In one embodiment the skin care product is a skin care product comprising an effective amount of the skin care composition described herein and one or more dermatologically or cosmetically acceptable components, wherein the one or more dermatologically acceptable component is a dermatologically acceptable carrier comprising about 10% to about 99% on a weight basis relative to a total weight of said skin care product.

In one embodiment the skin care product is a lotion, a serum, a jelly, a cream, a gel, an emulsion, a mask (such as but not limiting to a gel mask and a peel-off mask), a patch, or a stick comprising a dermatologically or cosmetically acceptable component and at least 1%, 2%, 3%, 4% up to 5% of a skin care composition described herein on a weight basis relative to a total weight of said skin care product.

In one embodiment the skin care product is a product comprising a combination of at least betaine, inositol, proline and serine in such a concentration as to provide an increased skin care benefit, when the skin care product is applied to a skin.

In one aspect the skin care product further comprises up to 25 wt. % of an antiperspirant, anti-microbial, a preservative, a fragrance, or any combination thereof.

A liquid skin care product may be aqueous, typically containing up to about 70% by weight of water and 0% to about 30% by weight of water soluble components.

Any number of dermatologically-acceptable materials commonly used in skin care products may also be incorporated into the present skin care products such as skin conditioning agents and skin colorants.

Skin conditioning agents as herein defined include, but are not limited to astringents, which tighten skin; exfoliants, which remove dead skin cells; emollients, which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Skin conditioning agents are well known in the art, see for example Green et al. (WO01/07009), and are available commercially from various sources. Suitable examples of skin conditioning agents include, but are not limited to, lactobionic acid, gluconic acid, alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils, xylitol, fucose, rhamnose, and triglyceride esters. The skin conditioning agents may include polysalicylates, propylene glycol (CAS No. 57-55-6, Dow Chemical, Midland, Mich.), glycerin (CAS No. 56-81-5, Proctor & Gamble Co., Cincinnati, Ohio), glycolic acid (CAS No. 79-14-1, DuPont Co., Wilmington, Del.), lactic acid (CAS No. 50-21-5, Alfa Aesar, Ward Hill, Mass.), malic acid (CAS No. 617-48-1, Alfa Aesar), citric acid (CAS No. 77-92-9, Alfa Aesar), tartaric acid (CAS NO. 133-37-9, Alfa Aesar), glucaric acid (CAS No. 87-73-0), galactaric acid (CAS No. 526-99-8), 3-hydroxyvaleric acid (CAS No. 10237-77-1), salicylic acid (CAS No. 69-72-7, Alfa Aesar), and 1,3 propanediol (CAS No. 504-63-2, DuPont Co., Wilmington, Del.). Polysalicylates may be prepared by the method described by White et al. in U.S. Pat. No. 4,855,483, incorporated herein by reference. Glucaric acid may be synthesized using the method described by Merbouh et al. (Carbohydr. Res. 336:75-78 (2001). The 3-hydroxyvaleric acid may be prepared as described by Bramucci in published international patent application number WO 02/012530.

Skin coloring agents may include the following dyes: eosin derivatives such as D&C Red No. 21 and halogenated fluorescein derivatives such as D&C Red No. 27, D&C Red Orange No. 5 in combination with D&C Red No. 21 and D&C Orange No. 10, and the pigments: titanium dioxide, titanium dioxide nanoparticles, zinc oxide, D&C Red No. 36 and D&C Orange No. 17, the calcium lakes of D&C Red Nos. 7, 11, 31 and 34, the barium lake of D&C Red No. 12, the strontium lake D&C Red No. 13, the aluminum lakes of FD&C Yellow No. 5, of FD&C Yellow No. 6, of D&C Red No. 27, of D&C Red No. 21, of FD&C Blue No. 1, iron oxides, manganese violet, chromium oxide, ultramarine blue, and carbon black. The coloring agent may also be a sunless tanning agent, such as dihydroxyacetone, that produces a tanned appearance on the skin without exposure to the sun.

Skin care products can comprise skin care additives such as, but not limiting to, colorants/dyes, fragrances, actives, preservatives, pH adjusters, chelators, and anti-oxidants.

In one embodiment the skin care product is a skin care lotion comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more components listed in Table 18, wherein said effective amount of the skin care composition is at least about 1%, 2%, 3%, 4%, 5% up to 10% on a weight basis relative to a total weight of said skin care product.

In one embodiment the skin care product is a skin care jelly comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more components listed in Table 19, wherein said effective amount of the skin care composition is at least about 1%, 2%, 3%, 4%, 5% up to 10% on a weight basis relative to a total weight of said skin care product

In one embodiment the skin care product is a skin care cream comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more components listed in Table 20, wherein said effective amount of the skin care composition is at least about 1%, 2%, 3%, 4%, 5% up to 10% on a weight basis relative to a total weight of said skin care product

In one embodiment the skin care product is a skin care gel comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more components listed in Table 21, wherein said effective amount of the skin care composition is at least about 1%, 2%, 3%, 4%, 5% up to 10% on a weight basis relative to a total weight of said skin care product.

In one embodiment the skin care product is a skin care emulsion an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more components listed in Table 22, wherein said effective amount of the skin care composition is at least about 1%, 2%, 3%, 4%, 5% up to 10% on a weight basis relative to a total weight of said skin care product.

In one embodiment the skin care product is a skin care stick comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more components listed in Table 23, wherein said effective amount of the skin care composition is at least about 1%, 2%, 3%, 4% up to 10% on a weight basis relative to a total weight of said skin care product.

In one embodiment the skin care product is a skin care gel mask comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more components listed in Table 24, wherein said effective amount of the skin care composition is at least about 1%, 2%, 3%, 4%, 5% up to 10% on a weight basis relative to a total weight of said skin care product.

In one embodiment the skin care product is a skin care cream comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more components listed in Table 25 or Table 26, wherein said effective amount of the skin care composition is at least about 1%, 2%, 3%, 4% up to 10% on a weight basis relative to a total weight of said skin care product.

In one embodiment, the skin care product is a skin care peel-off mask comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and one or more dermatologically or cosmetically acceptable components, wherein said effective amount of the skin care composition is at least about 1%, 2%, 3%, 4% up to 10% on a weight basis relative to a total weight of said skin care product. In one aspect the peel-off mask is made of a gel and an activating spray solution wherein the gel and/or activating spray solution contains an effective amount of the skin care composition described herein. The gel can be a clear or colored gel with optionally visible suspended natural ingredients like flower petals or natural beads. The active solution can be a clear or colored liquid with optionally visible flower heads or petals inside. In one aspect the peel-off mask comprises 4% of the skin care composition described herein (Example 1) on a weight basis relative to a total weight of said skin care product.

In one embodiment, the skin care product is a macro-emulsion cream comprising an effective amount of the skin care composition described herein (such as an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition) and about 1%, 2%, 3%, 4% up to 5% of GENENCARE® OSMS BA (DuPont Industrial Biosciences) and about 1%, 2%, 3%, 4% up to 5% GENENCARE® OSMS MI (DuPont Industrial Biosciences), wherein said effective amount of the skin care composition is at least about 1%, 2%, 3%, 4% up to 10% on a weight basis relative to a total weight of said skin care product. In one aspect the macro-emulsion cream comprises 1%, 2%, 3%, 4% up to 5% of the skin care composition described herein, 3% of GENENCARE® OSMS BA and 1% GENENCARE® OSMS MI on a weight basis relative to a total weight of said skin care product.

The skin care compositions and products described herein can also be part of a kit for providing one or more skin care benefits such as, but not limiting to, a kit for moisturizing the skin, a kit for osmoprotection of the skin (protecting the skin against the loss of substances endogenous to the body, e.g. water, lipids, electrolytes), a kit for protecting the skin against environmental stresses (such as chemicals, microorganisms), a kit for protecting the skin against pollution, a kit for protecting the skin against ultra violet irradiation damage, a kit for protecting the skin against blue light, a kit for protecting the skin against oxidative stress, a kit for skin detoxification, a kit for preventing or retarding the appearance of the signs of aging of the skin, a kit for reducing the appearance of skin wrinkles, a kit for skin rejuvenation, a kit for reducing dermal adhesions, a kit for promoting cell or tissue longevity, a kit for strengthening the skin barrier function, or any one combination thereof. In one aspect the kit is a kit for providing a skin care benefit, comprising a first component comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition; and a second component comprising an activating spray solution. In one aspect the kit is a kit for providing a skin care benefit, comprising an activating spray solution comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition; and a gel, wherein optionally said gel and activating spray solution can produce a peel-off mask.

### Contacting/Applying Skin Care Compositions and Skin Care Products

Effective amounts of skin care compositions described herein can be contacted with (applied to) the skin directly or as part of a skin care product.

In one aspect effective amounts of a skin care composition described herein or a skin care product comprising an effective amount of a skin care composition described herein is contacted with keratinocyte cells under hyperosmotic stress.

In one aspect an effective amount of a skin care composition described herein or a skin care product comprising an effective amount of a skin care composition described herein is contacted with keratinocyte cells under hyperosmotic stress and/or UV irradiation.

Skin care products can be contacted with (applied to) any surface of the skin. Skin care products can be topically applied to the skin and are referred to herein as topical skin care compositions.

In one aspect, skin care products are utilized on humans, while in other aspects, skin care products find use with non-human animals (e.g., in veterinary applications).

As used herein, “contacting” refers to placing an effective amount of a skin care composition described herein or a skin care product comprising an effective amount of a skin care composition described herein in contact with a skin (skin cell, skin surface). Contacting includes spraying, treating, immersing, flushing, pouring on or in, mixing, combining, painting, coating, applying, affixing to a skin care product described herein comprising a skin care composition described herein, solution with the skin surface.

Skin cell can be an in-vitro cell, an in-vivo cell, cell culture such as but not limiting to a human or animal skin cell.

### Methods for Providing an Increased Skin Care Benefit

The skin care products described herein can be used in methods for providing an increased skin care benefit.

In one embodiment the method is a method for providing an increased skin care benefit to a skin (skin surface, skin cell), said method comprising contacting a skin (skin surface, skin cell) with a skin care product comprising an effective amount of a skin care composition described herein. In one aspect the effective amount of the skin care composition described herein is at least 1%, 2%, 3%, 4% up to 5% of a skin care composition described herein on a weight basis relative to a total weight of said skin care product.

In one embodiment the method is a method for providing an increased skin care benefit to a skin, said method comprising contacting a skin with a skin care product comprising an effective amount of a skin care composition described herein, wherein said increased skin care benefit is selected from the group consisting of an increased osmoprotection, an increased protection from UV irradiation induced damage protection, or a combination thereof.

### Methods for Providing Multiple Skin Care Benefits

The skin care products described herein can be used in methods for providing multiple skin care benefit.

In one embodiment, the method is a method for providing multiple skin care benefits to a skin, said method comprising contacting a skin with a skin care product comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, wherein said multiple skin care benefits are at least two skin care benefits selected from the group consisting of skin moisturizing, osmoprotection of the skin, protection of the skin against pollution, protecting of the skin against ultra violet irradiation damage, protecting of the skin against blue light damage, protecting of the skin against oxidative stress, skin detoxification, strengthening the skin barrier function, and any one combination thereof.

Skin care benefits include, but are not limited to, osmoprotection, UV irradiation protection, blue light protection, night light protection, protection against oxidative stress, skin detoxification, improved skin barrier function, or any one combination thereof.

### General Definitions

The disclosures of all cited patent and non-patent literature are incorporated herein by reference in their entirety.

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “embodiment” or “disclosure” is not meant to be limiting, but applies generally to any of the embodiments defined in the claims or described herein. These terms are used interchangeably herein.

As used herein, the term “effective amount” refers to the amount of a given condition and/or composition, such as the skin care compositions described herein, subjected to a target material to obtain a desired effect (e.g., subjecting cells or skin to a skin care product comprising an amount of the skin care composition described herein to obtain at least one skin care benefit described herein). A desired effect include providing a skin care benefit, wherein the benefit is selected from the group consisting of skin moisturizing (protecting the skin against dehydration by maintaining, restoring and/or strengthening the moisturization of the skin), osmoprotection of the skin (protecting the skin against the loss of substances endogenous to the body, e.g. water, lipids, electrolytes), protecting the skin against environmental stresses (such as chemicals microorganisms), protecting the skin against pollution, protecting the skin against ultra violet irradiation damage, protecting the skin against blue light, protection against oxidative stress, skin detoxification, preventing or retarding the appearance of the signs of aging of the skin, reducing the appearance of skin wrinkles, skin rejuvenation, reducing dermal adhesions, promoting cell or tissue longevity, strengthening the skin barrier function, or any one combination thereof. In one aspect the effective amount of a skin care composition in a skin care product subjected to a skin can result in an increased benefit such as an increased osmoprotection, an increased protection of the skin against environmental stresses, an increased protection of the skin against ultra violet irradiation damage, or any one combination thereof.

In one aspect, the effective amount of a skin care composition described herein in a skin care product is at least about 1%, 2%, 3%, 4%, 5% up to 10% on a weight basis relative to a total weight of said skin care product.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values.

The terms “percent by weight”, “weight percentage (wt. %)” and “weight-weight percentage (% w/w)” are used interchangeably herein. Percent by weight refers to the percentage of a material on a mass basis as it is comprised in a composition, mixture, solution or product.

As used herein, the term “skin surface” refers to any surface of the skin that may serve as the target for a skin benefit agent. The presently claimed methods and compositions are directed to skin care applications and skin care products. As such, the skin surface comprises skin, preferably mammalian skin. In one embodiment, the skin surface is human skin.

As used herein, applying a skin care composition or a skin care product to a “skin” includes applying a skin care composition or a skin care product to a skin surface, to in-vitro or in-vivo skin cells, such as but not limiting to epidermal keratinocytes.

In one aspect the skin cells described herein are mammalian skin cells, such as human or animal skin cells.

As used herein, placing a skin care composition (or a skin care product comprising an effective amount of said composition) in contact with a skin surface includes placing a skin care composition or a skin care product in contact with in-vitro or in-vivo skin cells, such as but not limiting to epidermal keratinocytes.

The terms moisturizer, a lotion or a body lotion refer to a low to medium-viscosity emulsion of oil and water, most often oil-in-water but possibly water-in-oil with the primary benefit in a skin care application to hydrate the skin or to reduce its water loss. Nearly all moisturizers contain a combination of emollients, occlusives, and humectants. Emollients, which are mainly lipids and oils, hydrate and improve the appearance of the skin. A wide variety of suitable emollients is known and maybe used herein (International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (referred to as “ICI Handbook”) contains numerous examples of suitable materials). Occlusives such as petrolatum, lanolin and bee wax reduce transepidermal water loss by creating hydrophobic barrier over the skin. Humectants such as glycerol and urea able to attract water from the external environment and enhance water absorption from the dermis into the epidermis. In addition, the moisturizer formulations may contain emulsifiers to maintain stability of emulsions, and use thickeners to achieve desired viscosity and skin feel. A wide variety of other ingredients such as fragrances, dyes, preservatives, therapeutic agents, proteins and stabilizing agents are commonly added for other consumer preferred attributes.

As used herein, the term “biological contaminants” refers to one or more unwanted and/or pathogenic biological entities including, but not limited to, microorganisms, spores, viruses, prions, and mixtures thereof.

As used herein, the term “excipient” refers to inactive substance used as a carrier for active ingredients, in a formulation. The excipient may be used to stabilize the active ingredient in a formulation, such as the storage stability of the active ingredient. Excipients are also sometimes used to bulk up formulations that contain active ingredients. An “active ingredient” includes a skin care benefit agent as described herein.

Non-limiting examples of compositions and methods disclosed herein include:

1. A skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition.

2. A skin care composition comprising a combination of at least four osmolytes selected from the group consisting of proline, serine, betaine and inositol, wherein said combination comprises 30% proline, 14% serine, 50% betaine and 6% inositol on a % osmolyte basis relative to a total osmolyte content of 100%.

3. The skin care composition of any one of embodiments 1-2, further comprising at least one additional compound selected from the group consisting of preservatives, pH adjusters, anti-oxidants and chelators.

4. The skin care composition of embodiment 3, wherein the additional compound is a preservative.

5. The skin care composition of embodiment 4, wherein the preservative is sodium benzoate

6. The skin care composition of embodiment 3, wherein the additional compound is a pH adjuster.

7. The skin care composition of embodiment 6, wherein the pH adjuster is citric acid.

8a. The skin care composition of any preceding embodiments, wherein said skin care composition is a clear aqueous solution.

8b. The skin care composition of embodiments 1-8, wherein an effective amount of said composition in a formulation or skin care product provides a skin care benefit or multiple skin care benefits selected from the group consisting of a decrease in oxidative stress, an increased detoxification, and increased skin barrier function, or any one combination thereof.

8c. The skin care composition of embodiments 1-8, wherein an effective amount of said composition in a formulation or skin care product provides a skin care benefit or multiple skin care benefits selected from the group consisting of a reduction of lipid peroxidation, a reduction of UV-induced protein carbonylation, a reduction of UV-induced DNA damage, a reduction of ROS under UV exposure, an increase in oxidative stress transcription factors, an increase in anti-oxidant enzymes, a reduction of induced activation of MT-1H, a strengthening of the keratinocyte tight junctions, a decrease of caffeine diffusion, or any one combination thereof.

8d. The skin care composition of embodiments 8b and 8c, wherein the effective amount of said skin care composition is at least about 0.1% to 5% on a weight basis relative to a total weight of said formulation or skin care product.

9. Use of an effective amount of the skin care composition of any one preceding embodiment in a skin care product.

10. A skin care product comprising an effective amount of the skin care composition of any one of embodiment 1-8 and one or more dermatologically or cosmetically acceptable components.

11. The skin care product of embodiment 10, wherein said effective amount of the skin care composition is at least about 1%, 2%, 3%, 4% up to 5% on a weight basis relative to a total weight of said skin care product.

12. The skin care product of embodiment 10, wherein the one or more dermatologically acceptable component is a dermatologically acceptable carrier comprising about 10% to about 99% on a weight basis relative to a total weight of said skin care product.

13. A skin care product comprising a combination of the osmolytes proline, serine, betaine and inositol in a ratio of 30:14:50:6.

14a. The skin care product of embodiments 10-13, wherein said skin care product is selected from the group consisting of a lotion, a serum, a jelly, a cream, a gel, an emulsion, a mask, a face mask, a patch, and a stick.

14b. A skin care product or formulation comprising an effective amount of the skin care composition of embodiments 1-8, wherein said effective amount of said skin care composition provides a skin care benefit or multiple skin care benefits selected from the group consisting of a decrease in oxidative stress, an increased detoxification, and increased skin barrier function, or any one combination thereof.

14c. A skin care product or formulation comprising an effective amount of the skin care composition of embodiments 1-8, wherein said effective amount of said skin care composition provides a skin care benefit or multiple skin care benefits selected from the group consisting of a reduction of lipid peroxidation, a reduction of UV-induced protein carbonylation, a reduction of UV-induced DNA damage, a reduction of ROS under UV exposure, an increase in oxidative stress transcription factors, an increase in anti-oxidant enzymes, a reduction of induced activation of MT-1H, a strengthening of the keratinocyte tight junctions, a decrease of caffeine diffusion, or any one combination thereof.

14d. The skin care product or formulation of embodiments 14a-c, wherein the effective amount of the skin care composition is at least about 0.1% to 5% on a weight basis relative to a total weight of said formulation or skin care product.

15. A method for providing an increased skin care benefit to a skin, said method comprising contacting a skin with a skin care product comprising an effective amount of the skin care composition of any one of embodiments 1-8 and one or more dermatologically or cosmetically acceptable components.

16. The method of embodiment 15 wherein said increased skin care benefit is selected from the group consisting of an increased osmoprotection, an increased UV irradiation damages protection, increased blue light protection, and a combination thereof.

17. The method of embodiment 15, wherein said skin care product is a lotion, a serum, a jelly, a cream, a gel, an emulsion, a mask, a patch, or a stick comprising said a dermatologically acceptable carrier and at least 1%, 2%, 3%, 4% up to 5% of said skin care composition on a weight basis relative to a total weight of said skin care product.

18. A method for providing multiple skin care benefits to a skin, said method comprising contacting a skin with a skin care product comprising an effective amount a skin care composition of any one of embodiments 1-8, wherein said multiple skin care benefits are at least two skin care benefits selected from the group consisting of skin moisturizing, osmoprotection of the skin (protecting the skin against the loss of,), protection of the skin against pollution, protecting of the skin against ultra violet irradiation damage, protecting of the skin against blue light damage, protecting of the skin against oxidative stress, skin detoxification, strengthening the skin barrier function, and any one combination thereof.

19. A skin care lotion comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more components listed in Table 18, wherein said effective amount of the skin care composition is at least about 1%, 2%, 3%, 4%, 5% up to 10% on a weight basis relative to a total weight of said skin care product.

20. A skin care jelly comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more components listed in Table 19, wherein said effective amount of the skin care composition is at least about 1%, 2%, 3%, 4%, 5% up to 10% on a weight basis relative to a total weight of said skin care product

21. A skin care cream comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more components listed in Table 20, wherein said effective amount of the skin care composition is at least about 1%, 2%, 3%, 4%, 5% up to 10% on a weight basis relative to a total weight of said skin care product

22. A skin care gel comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more components listed in Table 21, wherein said effective amount of the skin care composition is at least about 1%, 2%, 3%, 4%, 5% up to 10% on a weight basis relative to a total weight of said skin care product

23. A skin care emulsion comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more components listed in Table 22, wherein said effective amount of the skin care composition is at least about 1%, 2%, 3%, 4%, 5% up to 10% on a weight basis relative to a total weight of said skin care product.

24. A skin care stick comprising an effective amount of an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more components listed in Table 23, wherein said effective amount of the skin care composition is at least about 1%, 2%, 3%, 5%, 4% up to 10% on a weight basis relative to a total weight of said skin care product.

25. A skin care gel mask comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more components listed in Table 24, wherein said effective amount of the skin care composition is at least about 1%, 2%, 3%, 4%, 5% up to 10% on a weight basis relative to a total weight of said skin care product.

26. A skin care night cream comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more components listed in Table 25 or Table 26, wherein said effective amount of the skin care composition is at least about 1%, 2%, 3%, 4% up to 10% on a weight basis relative to a total weight of said skin care product.

27. A skin care peel-off mask comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, and one or more dermatologically or cosmetically acceptable components, wherein said effective amount of the skin care composition is at least about 1%, 2%, 3%, 4% up to 10% on a weight basis relative to a total weight of said skin care product.

28. A macro-emulsion cream comprising an effective amount of a skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition, about 1%, 2%, 3%, 4% up to 5% of GENENCARE® OSMS BA (DuPont Industrial Biosciences) and about 1%, 2%, 3%, 4% up to 5% GENENCARE® OSMS MI (DuPont Industrial Biosciences), wherein said effective amount of the skin care composition is at least about 1%, 2%, 3%, 4% up to 10% on a weight basis relative to a total weight of said skin care product.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

The following abbreviations in the specification correspond to units of measure, techniques, properties, or compounds as follows: “sec” or “s” means second(s), “min” means minute(s), “h” or “hr” means hour(s), “μL” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “mg” means milligram(s), “μg” means microgram(s), “ng” means nanogram(s), “conc.” means concentration, “Trt” means treatment.

### Example 1

### Skin Care Compositions for Use in Skin Care Products

The osmolytes L-Proline, L-Serine, Betaine and Inositol were obtained in powder form and combined in a ratio of 30% L-Proline, 14% L-Serine, 50% Betaine, and 6% Inositol of total % osmolytes in an aqueous solution (Table 1).

TABLE 1
Skin care composition of Proline, Serine, Betaine and Inositol

| | Ratio of | |
|---|---|---|
| | osmolytes | |
| | (% of total | % of total |
| Osmolyte | osmolytes) | composition |
| L-Proline | 30 | 15 |
| L-Serine | 14 | 7 |
| Betaine | 50 | 25 |
| Inositol | 6 | 3 |
| Total | 100 | 50 |

In aspect the skin care composition further comprised the preservative sodium benzoate at 0.4 wt % and/or pH adjuster citric acid at 0.8 wt %

The skin care compositions can be used in formulations and skin care products as described herein having at least about 0.3% to 5.0% on a weight basis relative to a total weight of said formulation or skin care product.

### Example 2

### Osmolyte Combinations of Betaine, Inositol, Proline and Serine for Osmoprotection

To test the efficacy of compositions comprising the osmolytes Proline, Serine, Betaine and Inositol at a ratio of 30% Proline, 14% Serine, 50% Betaine, and 6% Inositol on a % osmolyte basis relative to a total osmolyte content of 100%, for use in a skin care product, two different products were prepared containing the same ratio of osmolytes but having different total osmolyte concentration. The combination C1 contained a total of 10 mg/ml osmolytes and the combination C2 contained a total of 3 mg/ml of osmolytes (Table 2).

TABLE 2
Skin care combinations C1 and C2

| | Ratio of | | |
|---|---|---|---|
| | osmolytes | Combination | Combination |
| | (% of total | C1 | C2 |
| Osmolyte | osmolytes) | (mg/ml) | (mg/ml) |
| L-Proline | 30 | 3.0 | 0.90 |
| L-Serine | 14 | 1.4 | 0.42 |
| Betaine | 50 | 5.0 | 1.50 |
| Inositol | 6 | 0.6 | 0.18 |
| Total | 100 | 10.0 | 3.00 |

Reference solutions were also prepared comprising either 3 mg/ml L-Proline as the sole osmolyte (referred to as L-Proline 3 mg/ml), or 10 mg/ml L-Proline as the sole osmolyte (referred to as L-Proline 10 mg/ml), or betaine at 10 mg/ml (referred to betaine 10 mg/ml).

### Example 3

### Osmoprotective Effects of Osmolyte Combinations on the Viability and Morphology of Keratinocyte Cells Under Stress and/or Irradiated Conditions

The osmolyte combinations C1 and C2 (Example 2) were tested for their osmoprotective effect on keratinocyte cells and compared to the effects of a reference composition comprising solely L-proline as an osmolyte (at either 3 mg/ml or 10 mg/ml).

### Biological Model

The type of cells use for this study were Normal Human Epidermal Keratinocytes (NHEK), Bioalternatives reference K341 used at the 3^{rd} passage. The culture conditions were: 37° C., 5% CO2. The culture medium is referred to as “keratinocyte-SFM” supplemented with Epidermal Growth Factor (EGF) 0.25 ng/ml, Pituitary extract (PE) 25 μg/ml, Gentamycin 25 μg/ml. The assay medium was “keratinocyte-SFM” supplemented with gentamycin 25 μg/ml.

### Culture and Treatment

Keratinocytes (NHEK) were seeded in 24-well plates and cultured for 24 hours in culture medium. The medium was then replaced by assay medium containing or not (control) the single osmolyte or the combination of osmolytes C1 or C2 and cells were pre-incubated for 24 hours.

After the Pre-Incubation:

For hyper-osmotic conditions, the medium was removed and replaced by assay medium containing NaCl at 150 mM and containing the single osmolyte or the combination of osmolytes (C1 or C2). The cells were then incubated for 48 hours. In parallel, a normo-osmotic control (no osmotic stress control) was performed.

For UV-irradiated conditions, the medium was then removed and replaced by assay medium and the cells were irradiated with UVB at 250 mJ/cm2 (+UVA at 1.6 J/cm2). The lamp used was a SOL500 Sun simulator equipped with an H2 filter (Dr. Hole, AG). A non-irradiated control condition was performed in parallel. After irradiation, the medium was removed and replaced by assay medium containing or not (control) the single osmolyte or the combination of osmolytes (C1 or C2) and the cells were incubated for 48 hours.

For UV-irradiated and hyper-osmotic conditions, the medium was then removed and replaced by assay medium and the cells were irradiated with UVB at 250 mJ/cm2 (+UVA at 1.6 J/cm2). The lamp used was a SOL500 Sun simulator equipped with an H2 filter (Dr. Höle, AG). After irradiation, the medium was removed and replaced by assay medium containing NaCl at 150 mM (hyperosmotic conditions) and containing the single osmolyte or the combination of osmolytes (C1 or C2) and the cells were then incubated for 48 hours. In parallel, a non-irradiated and normo-osmotic control was performed.

All experimental conditions were performed in n=3.

### Assessment/End Points

The cell cultures were observed with a microscope for morphological changes and analyzed by flow cytometer.

### Flow Cytometry Analysis

At the end of incubation, culture medium was removed and the cells were rinsed with a PBS solution. The cells were then detached using trypsin treatment and transferred into tubes. After several washes in PBS/BSA 0.2%, the cells were labeled with a propidium iodide (PI) solution (staining of dead cells).

Cell populations were then analyzed for number (total number of event per well during a fixed time), size (FSC), granularity (SSC) and viability parameters (% PI-positive cells, equivalent to % dead cells) by flow cytometry using BD FACSVerse™ flow cytometer.
Acquisition 1 represents the number of events (=number of cells) passing one by one in front of the cytometer laser over a fixed time. It doesn't distinct dead or living cells at this step. By subtracting the number of events (in hyperosmotic or irradiated condition) from the total number of events in normoosmotic conditions, it is possible to calculate the number of cells that have been lysed due to the stress conditions.
Acquisition 2, represents, on a fixed number of events, 1) the detection of the light scattered when the cells are crossing the laser, FSC-A, from which can be calculated the cell size, 2) the detection of the cells stained by Propidium Iodide (dead cells) from which can be calculated the % of viable cells.

### Assay Validation

To validate the assay, a treatment of the NHEK with NaCl at 150 mM for 48 hours was performed and resulted in a decrease of the total number of cells, cell size and viability (16%, 75% and 62% of the non-treated control under normo-osmotic condition, respectively). These results were expected and validated the assay.

Similarly, when NHEK were subjected to a combined stress (UVB irradiation+NaCl), there was a decrease of the total number of cells, cell size and viability (mean of 17%, 77% and 75% of the non-treated control under normo-osmotic and non-irradiated conditions).

### Results—Osmoprotective Effect of Osmolyte Combinations

The results of the assessment in control normo-osmotic conditions and in hyper-osmotic stress conditions, of the control and of the cells pre-incubated by L-Proline (3 mg/ml or 10 mg/ml) or by the C1 combination of osmolytes (10 mg/ml total osmolytes) on the cell number are shown in Table 3.

Table 4 shows the results of the assessment in control normo-osmotic conditions and in hyper-osmotic stress conditions, of the control and of the cells pre-incubated by L-Proline (3 mg/ml or 10 mg/ml) or by the combination of osmolytes C1-(10 mg/ml total osmolytes) or combination C2 (3 mg/ml total osmolytes) on the number of lysed cells.

Table 5 shows the results of the assessment in control normo-osmotic conditions and in hyper-osmotic stress conditions, of the control and of the cells pre-incubated by L-Proline (3 mg/ml or 10 mg/ml) or by the combination of osmolytes C1-(10 mg/ml total osmolytes) on cell size.

Table 6 shows the results of the assessment in control normo-osmotic conditions and in hyper-osmotic stress conditions, of the control and of the cells pre-incubated by L-Proline (3 mg/ml or 10 mg/ml) or by the combination of osmolytes C1-(10 mg/ml total osmolytes on cell survival.

The results are expressed either in % of hyperosmotic control, or, for the combinations (C1, C2) also in % of L-Proline (3 mg/ml and/or 10 mg/ml), with indication of the level in the significant differences when the effect of the combinations are significantly superior than that of L-Proline. The legend for the significant differences are as follows: ns: >0.05, non significant; *: 0.01 to 0.05, Significant; **: 0.001 to 0.01, very significant and ***: <0.001, Extremely significant.

TABLE 3
Effect of osmolyte combinations on the cell number in hyperosmotic stress conditions.

| | | | Acquisition 1 (Fixed time) | | | | | | |
|---|---|---|---|---|---|---|---|---|---|
| | | | Total number of events per well | | | | | | |
| | Treatment | | | % | | % | P * vs | % | P * vs |
| | Test | Conc. | Mean | hyperosmotic | P * vs | L-Proline | L-proline | L-Proline | L-proline |
| | compound | mg/ml | (au) | control | control | (3 mg/ml) | (3 mg/ml) | (10 mg/ml) | (10 mg/ml) |
| Normo-osmotic control | | — | 91873 | 640 | *** | — | — | — | — |
| Hyperosmotic | Control | | 14351 | 100 | — | — | — | — | — |
| conditions | L-Proline | 3 | 18676 | 130 | — | — | — | — | — |
| NaCl 150 mM | L-Proline | 10 | 34878 | 243 | — | — | — | — | — |
| | Osmolyte | 10 | 73042 | 509 | *** | 391 | *** | 209 | ** |
| | combination C1 | | | | | | | | |

TABLE 4
Effect of osmolyte combinations as measured by number of lysed cells.

| | | | Lysed cells | |
|---|---|---|---|---|
| | | | Mean number of event per well | |
| | Treatment | | normo-osmotic control − | % normo- |
| | Test | Conc. | Mean number of event per | osmotic |
| | compound | mg/ml | well hyperosmotic conditions | control |
| Normo-osmotic control | | — | — | 100.0 |
| Hypeosmotic | Control | | 77522 | 84.4 |
| conditions: | L-Proline | 3 | 73197 | 67.2 |
| NaCl 150 | L-Proline | 10 | 56995 | 41.7 |
| mM | Osmolyte | 10 | 18831 | 7.5 |
| | combination C1 | | | |

TABLE 5
Effect of osmolyte combinations on cell size in hyperosmotic stress conditions.

| | | | Acquisition 2 (Fixed number of events) | | |
|---|---|---|---|---|---|
| | Treatment | | Cell size | | |
| | Test | Conc. | Mean cell size | % hyperosmotic | P * vs. |
| | compound | (mg/ml) | FSC-A (au) | control | control |
| Normo-osmotic control | | — | 87844 | 133 | *** |
| Hyperosmotic | Control | | 65928 | 100 | — |
| conditions: | L-Proline | 3 | 68417 | 104 | ns |
| NaCl 150 | L-Proline | 10 | 74581 | 113 | * |
| mM | Osmolyte | 3 | 72967 | 111 | *** |
| | combination C2 | | | | |
| | Osmolyte | 10 | 76578 | 116 | *** |
| | combination C1 | | | | |

TABLE 6
Effect of osmolyte combinations on cell viability (survival) in hyperosmotic stress conditions.

| | | | Mean % viable | | | | |
|---|---|---|---|---|---|---|---|
| | | | cells = 100-% | | | | |
| | | | PI-positive | % | | % | P* vs |
| | | Conc. | cells = 100-% | hyperosmotic | P* vs | L-Proline | L-proline |
| Test compound | | (mg/ml) | dead cells | control | control | 3 mg/ml | 3 mg/ml |
| Normo-osmotic control | | — | 78.7 | 161 | * | — | — |
| Hyperosmotic | Control | | 48.8 | 100 | — | — | — |
| conditions: | L-Proline | 3 | 69.6 | 143 | * | — | — |
| NaCl 150 mM | Osmolyte | 10 | 93.2 | 191 | ** | 134 | *** |
| | combination C1 | | | | | | |

The results in Tables 2-5 showed that the osmolyte combination of C1 (comprising L-Proline+L-Serine+Betaine+Inositol at a total osmolyte concentration of 10 mg/ml) showed an unexpectedly and surprisingly high protection against hyper-osmotic stress. Under the hyper-osmotic conditions with NaCl at 150 mM, combination L-Proline+L-Serine+betaine+inositol, tested at the highest concentration (10 mg/ml), induced a protective effect against hyper-osmotic stress by strongly increasing the number of cells (509% of the hyper-osmotic control—Table 3), strongly decreasing the % of cell lysis (from 84.4% to 7.5%—Table 4), strongly increasing the cell size (116% of the hyper-osmotic control—Table 5) and strongly increasing the cell size cell viability (191% of the hyper-osmotic control—Table 6).

Moreover, as illustrated in Table 7, this combination of osmolytes prevents the decrease of cell size compared to hyperosmotic stress conditions without osmolytes further supporting the unexpected and surprising protective effect against.

TABLE 7
Effect of osmolyte combinations on cell
size in hyperosmotic stress conditions.

| | % cell size difference compared |
|---|---|
| Test compound (concentration | to control hyperosmotic stress |
| of total osmolytes) | condition under 150 mM NaCl |
| L-Proline (3 mg/ml) | +4% |
| Combination C2 (3 mg/ml) | +11% |
| L-Proline (10 mg/ml) | +13% |
| Combination C1 (10 mg/ml) | +16% |

The magnitude of the osmoprotection obtained from the C1 combination is unexpected and surprising when compared to the effect of a composition comprising the same total concentration of osmolytes (such as L-Proline 10 mg/ml). As shown in Table 3, the effect of the C1 combination on cell number is significantly stronger (209% higher when compared to the effect of L-Proline at 10 mg/ml. Furthermore, as shown in Table 4 the effect of the C1 combination the number of lysed cells was also significantly greater (7.5% cell lysis observed for the C1 combination versus 41.7% lysis for L-Proline at 10 mg/ml) indicating a 34.2% reduction in cell lysis.

It is also unexpected when one compares the effect of the C1 composition (which comprises 3 mg/ml L-Proline) with the L-Proline reference composition at 3 mg/ml. As shown in Table 3, the effect of the C1 combination on cell number is significantly stronger (391% higher when compared to the effect of L-Proline at 3 mg/ml. Also as shown in Table 4 the effect of the C1 combination on the number of lysed cells was also significantly greater (7.5% cell lysis observed for the C1 combination versus 67.2% lysis for L-Proline at 10 mg/ml) indicating a 59.7% reduction in cell lysis. Under the hyper-osmotic conditions with NaCl at 150 mM, combination L-Proline+L-Serine+Betaine+Inositol, tested at the concentration of 3 mg/ml (C2) significantly increased the cell size (111% of the hyper-osmotic control—Table 5), whereas L-Proline at the same concentration 3 mg/ml didn't significantly increase the cell size. The C2 combination at 10 mg/ml (comprising 3 mg/ml L-proline) also showed a significantly higher protective effect on cell viability (134% of the L-Proline 3 mg/ml) than L-Proline at 3 mg/ml

### Results—Protection of Osmolyte Combinations Against UVB Irradiation

The results of the assessment in non-irradiated control conditions and non-irradiated & normo-osmotic control conditions, of the control and of the cells pre-incubated by L-Proline (3 mg/ml or 10 mg/ml) or by the C1 combination of osmolytes (10 mg/ml total osmolytes) on cell size are shown in Table 8.

TABLE 8
Effect of osmolyte combinations on cell size in irradiated stress conditions.

| | | | Acquisition 2 (Fixed number of events) | | |
|---|---|---|---|---|---|
| | Treatment | | Cell size | | |
| | Test | Conc. | Mean cell size | % hyperosmotic | P * vs |
| | compound | (mg/ml) | FSC-A (au) | control | control |
| Non-irradiated control | | — | 87844 | 96 | ns |
| UVB | Control | | 91624 | 100 | — |
| 250 mj/cm² | L-Proline | 3 | 100519 | 110 | ns |
| | L-Proline | 10 | 101243 | 110 | ns |
| | Osmolyte | 10 | 103080 | 113 | * |
| | combination C1 | | | | |
| Non-irradiated & | | | 87844 | 129 | *** |
| normo-osmotic control | | | | | |
| Hyperosmotic | Control | — | 68061 | 100 | — |
| stress NaCl | L-Proline | 3 | 69601 | 102 | ns |
| 150 mM + UVB | L-Proline | 10 | 68169 | 100 | ns |
| 250 mj/cm² | Osmolyte | 10 | 71262 | 105 | * |
| | combination C1 | | | | |

Table 9 shows the results of the assessment in non-irradiated control conditions, of the control and of the cells pre-incubated by L-Proline (3 mg/ml or 10 mg/ml) or by the combination of osmolytes C1 (10 mg/ml total osmolytes) on cell survival.

TABLE 9
Effect of osmolyte combinations on cell survival in irradiated stress conditions.

| | | | Acquisition 2 (Fixed number of events) | | | | |
|---|---|---|---|---|---|---|---|
| | | | Cell viability | | | | |
| | | | Mean % viable | | | | |
| | | | cells = 100-% | | | | |
| Treatment | | | PI-positive | % | | % | P* vs |
| | | Conc. | cells = 100-% | hyperosmotic | P* vs | L-Proline | L-proline |
| Test compound | | mg/ml | dead cells | control | control | 3 mg/ml | 3 mg/ml |
| Non-irradiated control | | — | 78.7 | 94 | ns | — | — |
| UVB | Control | | 83.6 | 100 | — | — | — |
| 250 mj/cm² | L-Proline | 3 | 86.4 | 103 | ns | — | — |
| | Osmolyte | 10 | 89.3 | 107 | ** | 103 | * |
| | combination C1 | | | | | | |

When the cells were submitted to UVB irradiation alone, 250 mJ/cm², or in combination with hyperosmotic stress, no clear effect was observed with L-Proline (Table 8). One could note a stimulating effect on the cell size and cell viability (respectively 113%, and 107% of the irradiated control, Table 8 and Table 9 respectively) of the combination C2 (10 mg/ml) in response to 250 mJ/cm² of UVB. As shown in Table 9, the effect of the combination C2 on cell survival is significantly higher (+103% of the irradiated control) than the effect of L-Proline 3 mg/ml.

The osmolyte combination C2 also showed a protective effect against the combination of UVB (250 mJ/cm²) and hyperosmotic stress (NaCl 150 mM) with a cell size increase+105% of hyperosmotic and irradiated control (Table 8).

### Example 4

### Caffein Diffusion Test on Reconstituted Human Epidermis (RHE)

The epidermis provides a physical and permeability barrier. This barrier is continuously regenerated by terminally differentiating keratinocytes, a process known as cornification or epidermal differentiation. In the epidermis there are multiple components important to barrier function including proteins (i.e. claudin, desmoglein, filaggrin, etc.), lipids (i.e. ceramides) and proteases. When properly functioning, the epidermis prevents water loss and provides a barrier to allergen and bacteria invasion.

Caffein is an ingredient being increasingly used in cosmetics due to its activity and ability to penetrate the skin barrier. As such, Caffein is used as a reference molecule for evaluating the skin barrier permeability, also called strength. Epiderm is the main barrier of the skin and Reconstructed Epidermis Models (RHE) are used to replicate in-vitro the main skin barrier characteristics.

The diffusion of topically applied caffein through reconstructed epidermis represents a suitable method to investigate the capacity of a compound to reinforce the epidermal barrier function. The stronger the epiderm structure and barrier function will be, the less caffein will be able to diffuse through it. The caffeine diffusion will be slower or limited by a stronger barrier.

In this example we describe the evaluation of the effects of osmolyte combinations (combination C1 and C2) and reference osmolyte compositions (L-serine at 3 mg/ml or 10 mg/ml; betaine at 10 mg/ml)) on the epiderm barrier function by following the diffusion of radioactive caffein ([¹⁴C]caffein) through the RHE after it is deposited topically on the epiderm (RHE) surface. By measuring the amount of caffein in the subnatant (cpm—count per minute), one can calculate the caffein diffusion, and then translate this data into barrier function value (% control).

### Biological Model

The epidermis used was a Reconstructed Human Epidermis, called “RHE”, 0-day-old, batch no 01015-444, placed in culture conditions 37° C., 5% CO2. The assay medium was the Bioalternatives maintenance medium.

### Culture and Treatment

RHE at Day 0 (when placed at the air/liquid interface) were placed in assay medium containing or not (control) the compounds, the combination or the reference (vitamin C at 200 μg/ml). This is referred to as a systemic application, to be differentiated from a topical application. RHE were then incubated for 7 days with treatment renewal at Day 3 and Day 5. All experimental conditions were performed in n=3.

### Caffein Diffusion

At the end of incubation (Day 7), radioactive caffein ([¹⁴C]caffein) was applied on the surface of each epidermis. The radioactivity contained in subnatants was measured by liquid scintillation after 30 minutes, 1, 2, 3, 4, 5, 6, 7 and 8 hours of incubation.

### Barrier Function Calculation

The barrier function is calculated according to the below formula:

$$Barrier {function}_{( \% Control )} = \frac{Mean [ \frac{1000}{{cpm}_{sample}} ]}{Mean [ \frac{1000}{{cpm}_{Control}} ]} \times 100$$

### Assay Validation

Caffein diffusion through control reconstructed human epidermis (RHE) under non-treated condition was progressive during the whole kinetic. The systemic treatment of RHE with vitamin C, tested at 200 μg/ml, decreased significantly this diffusion, thus highlighting an increase of epidermal barrier function. These results were expected and validated the assay.

### Results

The results are shown in Table 10 and indicate that under the experimental conditions of the assay, the systemic treatment of the RHE with the osmolyte combinations of L-Proline+L-Serine+Betaine+Inositol, tested at 3 and 10 mg/ml (respectively C2 and C1), induced a decrease of caffein diffusion during the two first hours with a statistically significant effect at 30 minutes and 1 hour.

The systemic treatment of the RHE with a composition comprising only the single osmolyte Betaine, tested at 3 mg/ml, induced a decrease of caffein diffusion through the RHE only after 30 minutes. No inhibitory effect was observed at later times of incubation or in response to a stronger concentration (10 mg/ml). The systemic treatment of the RHE with a composition comprising only the single osmolyte L-Proline, tested at 3 and 10 mg/ml, did not decrease caffein diffusion through the RHE.

Moreover, one can notice that at every time point, the effect on the barrier function of the combination of osmolytes is significantly higher than L-Proline.
The combination C2 (3 mg/ml) is performing better than L-Proline (3 mg/ml) at 30 min, 1 h, 2 h and 3 hours and also better than L-Proline (10 mg/ml) the first 30 min and 1 hours.
The combination C1 (10 mg/ml) is performing better L-Proline (3 mg/ml) at every time point until 8 hours (end of the test) and better than L-Proline (10 mg/ml) until 4 hours (included).
The combinations C1 and C2 are also significantly performing better than betaine (10 mg/ml) at 2, 3 and 5 hours.

TABLE 10
Caffein diffusion test on RHE

| | | | | | | | Barrier | | Barrier | |
|---|---|---|---|---|---|---|---|---|---|---|
| | | | Mean | | Barrier | | function | | function | |
| | | | [14C]- | | function | | (% | *p vs | (% | *p vs |
| Trt | Test | Conc. | caffein | % | % | *p vs | L-Proline | L-Proline | L-Proline | L-Proline |
| (hrs) | compound | mg/ml | (cpm) | Control | Control | control | 3 mg/ml) | (3 mg/ml) | 10 mg/ml) | (10 mg/ml) |
| 0.5 | control | | 19667 | 100 | 100 | — | — | — | — | — |
| | L-Proline | 3 | 20720 | 105 | 96 | ns | — | — | — | — |
| | L-Proline | 10 | 20193 | 103 | 98 | ns | — | — | — | — |
| | betaine | 10 | 20507 | 104 | 97 | ns | — | — | — | — |
| | Osmolyte | 3 | 15667 | 80 | 126 | ** | 132 | * | 129 | * |
| | combination C2 | | | | | | | | | |
| | Osmolyte | 10 | 12880 | 65 | 154 | *** | 161 | ** | 157 | ** |
| | combination C1 | | | | | | | | | |
| 1 | control | | 45595 | 100 | 100 | — | — | — | — | — |
| | L-Proline | 3 | 47352 | 104 | 97 | ns | — | — | — | — |
| | L-Proline | 10 | 45495 | 100 | 100 | ns | — | — | — | — |
| | betaine | 10 | 50356 | 110 | 91 | ns | — | — | — | — |
| | Osmolyte | 3 | 38973 | 85 | 117 | * | 121 | * | 117 | ** |
| | combination C2 | | | | | | | | | |
| | Osmolyte | 10 | 33685 | 74 | 135 | ** | 140 | ** | 135 | ** |
| | combination C1 | | | | | | | | | |
| 2 | control | | 100261 | 100 | 100 | — | — | — | — | — |
| | L-Proline | 3 | 106026 | 106 | 94 | ns | — | — | — | — |
| | L-Proline | 10 | 102027 | 102 | 98 | ns | — | — | — | — |
| | betaine | 10 | 117502 | 117 | 85 | * | — | — | — | — |
| | Osmolyte | 3 | 99321 | 99 | 101 | ns | 107 | ns | 103 | ns |
| | combination C2 | | | | | | | | | |
| | Osmolyte | 10 | 90817 | 91 | 110 | ns | 117 | ** | 112 | ** |
| | combination C1 | | | | | | | | | |
| 3 | control | | 136851 | 100 | 100 | — | — | — | — | — |
| | L-Proline | 3 | 147545 | 108 | 93 | * | — | — | — | — |
| | L-Proline | 10 | 140203 | 102 | 98 | ns | — | — | — | — |
| | betaine | 10 | 156579 | 114 | 87 | ** | — | — | — | — |
| | Osmolyte | 3 | 135820 | 99 | 101 | ns | 109 | * | 103 | ns |
| | combination C2 | | | | | | | | | |
| | Osmolyte | 10 | 129804 | 95 | 105 | ns | 114 | *** | 108 | * |
| | combination C1 | | | | | | | | | |
| 4 | control | | 157896 | 100 | 100 | — | — | — | | — |
| | L-Proline | 3 | 169001 | 107 | 93 | ns | — | — | — | — |
| | L-Proline | 10 | 162571 | 103 | 97 | ns | — | — | — | — |
| | betaine | 10 | 173278 | 110 | 91 | ns | — | — | — | — |
| | Osmolyte | 3 | 161148 | 102 | 98 | ns | 105 | ns | 101 | ns |
| | combination C2 | | | | | | | | | |
| | Osmolyte | 10 | 154614 | 98 | 102 | ns | 109 | ** | 105 | * |
| | combination C1 | | | | | | | | | |
| 5 | control | | 172361 | 100 | 100 | — | — | — | — | — |
| | L-Proline | 3 | 184911 | 107 | 93 | ns | — | — | — | — |
| | L-Proline | 10 | 176461 | 102 | 98 | ns | — | — | — | — |
| | betaine | 10 | 186588 | 108 | 92 | * | — | — | — | — |
| | Osmolyte | 3 | 177443 | 103 | 97 | ns | 104 | ns | 99 | ns |
| | combination C2 | | | | | | | | | |
| | Osmolyte | 10 | 171809 | 100 | 100 | ns | 108 | * | 103 | ns |
| | combination C1 | | | | | | | | | |
| 6 | control | | 180285 | 100 | 100 | ns | — | — | — | — |
| | L-Proline | 3 | 190819 | 106 | 94 | * | — | — | — | — |
| | L-Proline | 10 | 181674 | 101 | 99 | ns | — | — | — | — |
| | betaine | 10 | 193499 | 107 | 93 | * | — | — | — | — |
| | Osmolyte | 3 | 187859 | 104 | 96 | ns | 102 | ns | 97 | ns |
| | combination C2 | | | | | | | | | |
| | Osmolyte | 10 | 180863 | 100 | 100 | ns | 106 | * | 100 | ns |
| | combination C1 | | | | | | | | | |
| 7 | control | | 188471 | 100 | 100 | — | — | — | — | — |
| | L-Proline | 3 | 199859 | 106 | 94 | ns | — | — | — | — |
| | L-Proline | 10 | 190843 | 101 | 99 | ns | — | — | — | — |
| | betaine | 10 | 196233 | 104 | 96 | ns | — | — | — | — |
| | Osmolyte | 3 | 195326 | 104 | 96 | ns | 102 | ns | 98 | ns |
| | combination C2 | | | | | | | | | |
| | Osmolyte | 10 | 188645 | 100 | 100 | ns | 106 | * | 101 | ns |
| | combination C1 | | | | | | | | | |
| 8 | L-Proline | 3 | 193655 | 100 | 100 | — | — | — | — | — |
| | L-Proline | 10 | 203011 | 105 | 95 | ns | — | — | — | — |
| | betaine | 10 | 195439 | 101 | 99 | ns | — | — | — | — |
| | Osmolyte | 3 | 201481 | 104 | 96 | ns | — | — | — | — |
| | combination C2 | | | | | | | | | |
| | Osmolyte | 10 | 200274 | 103 | 97 | ns | 101 | ns | 98 | ns |
| | combination C1 | | | | | | | | | |
| | L-Proline | 3 | 194941 | 101 | 99 | ns | 104 | * | 100 | ns |

As shown in Table 10, the barrier function of C1 at 30 minutes was 154% versus the control at 100%, indicating that the caffeine diffusion decreased by 54% after 30 minutes. Furthermore, as shown in Table 10, the osmolyte combination C1 (which is representative of a formulation or product comprising an effective amount of the skin care composition of example at 1 wt %) contributes to a decreased caffeine diffusion during the first four hours and as such helps improve the skin barrier function.

Further evidence that an effective amount of the skin care composition of example 1 can help improve the skin's barrier function was obtained from the measurement of Trans Epithelial Electrical Resistance (TEER) on keratinocytes cultures (NHEK) with chopstick electrodes. Tight junctions are keratinocyte's cell-cell junction in the stratum granulosum. The strength of Tight Junctions is related to the skin barrier function. Tight junction strength was tested in normal human epidermal keratinocyte cultures cultivated in cell cultivation inserts. The differentiation of the cells, and the formation of the tight junctions between the cells was induced with a well-known method by applying 1.45 mM CaCl₂. Test solutions were added on third day after initiation of differentiation, and the strength of tight junctions was measured with the chopstick electrodes. Application of an effective amount of the skin care composition of example 1 at 1 wt % resulted in a % TEER of 28% (at 2 hours), 28% (at 3 hours) and 43% (at 5 hours) versus a medium control not comprising the 1% skin care composition (having −5%, −7% and 14%, respectively). This data indicated that a 29% TEER increase (43% versus 14%) after 5 hours, was observed at an effective amount of the skin care composition. This data further indicated that an effective amount of the skin care composition of example 1 contributes to increased tight junction strength, thereby helping improving the skin's barrier function.

### Example 5

### Assessment of the Anti-Pollution Activity of Two Products on Living Human Skin Explants Ex-Vivo

### Products

Products were stored at room temperature during the study.

Product A (Table 11) was diluted in water to obtain the following products at concentrations: product P1=A at 4%, product P2=A at 2% and product P3=A at 1%.

Product B was diluted in water to obtain the following products at concentrations: product P4=B at 1% (10 mg/ml), product P5=B at 0.5% (5 mg/ml) and product P6=B at 0.3% (3 mg/ml).

TABLE 11
Product A

| | % of total |
|---|---|
| Ingredient | composition |
| Betaine | 25 |
| L-Proline | 15 |
| L-Serine | 7 |
| Inositol | 3 |
| Water | 50 |

### Product B: L-Proline

### Explant Preparation

59 human skin explants of an average diameter of 14 mm (±1 mm) were prepared on an abdoplasty coming from a 47-year-old caucasian woman. The explants were kept in survival in BEM culture medium at 37° C. in a humid, 5%-CO2 atmosphere. The explants were distributed into 5 batches as shown in Table 12.

TABLE 12
Explant batches

| | | Pollutants | Number of | Sampling |
|---|---|---|---|---|
| Batch | Treatment | exposure | explants | time |
| T0 | — | − | 3 | Day 0 |
| T | — | − | 4 | Day 5 |
| P1J5 | Product A at 4% | − | 4 | Day 5 |
| P2J5 | Product A at 2% | − | 4 | Day 5 |
| P4J5 | Product B at | − | 4 | Day 5 |
| | 10 mg/mL (1%) | | | |
| P5J5 | Product B at | − | 4 | Day 5 |
| | 5 mg/mL (0.5%) | | | |
| P6J5 | Product B at | − | 4 | Day 5 |
| | 3 mg/mL (0.3%) | | | |
| ML | — | + | 4 | Day 5 |
| P1MLJ5 | Product A at 4% | + | 4 | Day 5 |
| P2MLJ5 | Product A at 2% | + | 4 | Day 5 |
| P4MLJ5 | Product B at | + | 4 | Day 5 |
| | 10 mg/mL (1%) | | | |
| P5MLJ5 | Product B at | + | 4 | Day 5 |
| | 5 mg/mL (0.5%) | | | |
| P6MLJ5 | Product B at | + | 4 | Day 5 |
| | 3 mg/mL (0.3%) | | | |

### Pollutant

TABLE 13
Product descriptions

| Product | Identification | Reference | Aspect |
|---|---|---|---|
| A | Solution ICP multi | 1.10714.050 | liquid |
| | elements standard V | 0 (Merck) | |
| B | Benzene | 12550 | liquid |
| | | (Fluka) | |
| C | Xylene | 95673 | liquid |
| | | (Fluka) | |
| D | Toluene | 34866 | liquid |
| | | (Sigma) | |

The solution A (ICP multi-element standard V) is a mixture of heavy metals supplemented with hydrocarbons (B+C+D). The composition and concentration of each component of this pollutant mixture are indicated below:

Final concentrations applied on the explants of each compound of ICP multi-element standard V Certi Pur® solution (Merck; reference 1.10714.0500; batch HC309202) supplemented with hydrocarbons and diesel particles:

### Heavy Metals

Al 0.01 mg/mL
AS 0.01 mg/mL
B 0.001 mg/m L
Ba 0.001 mg/mL
Be 0.0005 mg/mL
Ca 0.005 mg/mL
Cd 0.001 mg/mL
Cr 0.001 mg/mL
Cu 0.001 mg/mL
Fe 0.001 mg/mL
Hg 0.0025 mg/mL
K 0.0495 mg/mL
Li 0.001 mg/mL
Mg 0.0005 mg/mL
Mn 0.0005 mg/mL
Na 0.01 mg/mL
Ni 0.0025 mg/mL
P 0.005 mg/mL
Pb 0.01 mg/mL
Sc 0.0005 mg/mL
Sa 0.01 mg/mL
Sr 0.0005 mg/mL
Te 0.01 mg/mL
Ti 0.001 mg/mL
Y 0.0005 mg/mL
Zn 0.001 mg/mL

### Hydrocarbons:

### Benzene 1 μL/mL

### Xylene 1 μL/mL

Toluene 1 μl/mL

### Explants Treatment

On day 0, explants were placed in 2 mL of culture medium with or without the products P1 to P6. Controls did not receive any treatment.

The tested products were topically applied on the basis of 2 μl per cm² and spread using a small spatula twice a day (morning and evening) on day 0, 1, 2, 3 and 4.

The culture medium was half-refreshed on day 2 (1 mL/well) and fully-refreshed on day 4 (2 mL/well) after pollutants exposure.

### Exposition of Skin Explants to Pollutants

On D4, the explants of the batches ML and PML were placed on the 3D grid with 900 μl of HBSS and placed into the PolluBox® system.

3 mL of the solution <<ML>> (supplemented with 150 μl/ml of NaCl 0.9%) were exposed by vaporization for 1 hour and half, as shown in the figure below.

During the exposition, the explants of the batches T and P were placed in a 12-well culture plate with 900 μl of HBSS solution per well.

At the end of the exposure, all the explants were put back in the incubator under standard culture conditions in 2 ml of fresh BEMc per well.

### Sampling

On D0, the 3 explants from the batch TO were collected and cut in two parts. Half was fixed in buffered formalin and half was frozen at −80° C.

On Day 5, 4 explants of all batches were collected and treated in the same way as on D0, and 2 ml of culture medium from all batches were collected and stored at −80° C. for MDA.

### Histological Processing

After fixation for 24 hours in buffered formalin, the samples were dehydrated and impregnated in paraffin using Leica TP1020 dehydration automat according to the document MO-H-149. The samples were embedded using a Leica EG 1160 embedding station.

5-μm-thick sections were made using a Leica RM 2125 Minot-type microtome, and the sections were mounted on Superfrost® histological glass slides.

The microscopical observations were realized using a Leica DMLB or Olympus BX43 microscope. Pictures were digitized with a numeric DP72 Olympus camera and analyzed with CellD storing software (Olympus Microscopy, Matrix Optics (M)SdnBhd, Taman Mayhang, Maylasia).

### Assessment/End Points

Biochemical Assay of MDA

Malondialdehyde (MDA) is a product arising from lipid peroxidation of cell membranes. The free radicals induced by oxidative stress (UVA, pollutants, heavy metals, pesticides, etc.) degrade the polyunsaturated lipids and generate hydroperoxides resulting in the formation of radical intermediates and aldehydes, particularly MDA (Janero. Free Radic Bioi Med. 1990; 9(6}: 515-40).

The MDA assay was realized with an enhanced method of the TBARs (Thiobarbituric Acid Reactive Species) assay. The MDA was assayed in HBSS medium by addition of TBARs solution (thiobarbituric acid, hydrochloric acid and tricholoroacetic acid) and placed in a water bath (80° C. for 15 minutes). A lot of substances (like glucose) which are not related with lipoperoxidation, react with thiobarbituric acid (ThioBarbituric Acid Reagents=TBARS), so to enhance the specificity of the assay, the MDA was extracted by a liquid/liquid extraction with butanol. The MDA in butanol was measured in spectrofluorimetry (excitation: 515 nm, emission: 550 nm) using a Tecan Infinite M200 Pro microplate reader. Using this assay, MDA contained in the culture medium BEMc on day 3 was measured for the 4 explants per batch. The MDA concentration was expressed in pmol of MDA/ml of the medium. Concerned batches: all batches on day 5, 4 explants per batch.

Immunostaining of Nrf2

Nrf2 (nuclear factor erythroid 2-related factor) is a transcription factor in cellular response to oxidative stress. Under oxidative stress, Nrf2 is activated, dissociated from its inhibitor (Keap1), translocated to the nucleus where it binds to the DNA at the ARE location (Antioxidant response element, a master regulator of the total anti-oxidant system). Nrf2 plays a role in protecting human skin keratinocytes from UVA radiation-induced damages (Tia et al. 2011. Bioscience Trends 5:23-29). Nrf2 induction was assayed by immunostaining in the living epidermis of human skin explant as follows: Nrf2 immunostaining was performed on paraffinized sections with a monoclonal antibody anti-Nrf2 (abcam, ref. ab76026, clone EP1809Y), diluted 1:200 in PBS-BSA 0.3%-Tween 20 0.05% during 1 h at room temperature with the system Vectastain RTU Universal VECTOR streptavidine/biotine and revealed with VIP (Vector laboratories, SK4600), a violet substrate of peroxydase. The immunostaining was performed using an automated slide processing system (Autostainer, Dako) and assessed by microscopical observation. Concerned batches: all the batches

Immunostaining of AHR

Aryl hydrocarbon receptor (AhR) is a transcriptional factor implicated in molecular response following the exposure of several compounds, including aromatic polycyclic hydrocarbon and ozone (Haarmann-Stemmann et al. 2012.JID 132:7-9; Afaq et al., 2009 JID 129:2396-2403). AhR is involved in activation of cytochrome family genes and CYP450 detoxification enzymes. In presence of pollution AhR is dissociated from the protein complex (HSP90), translocated to the nucleus, dimerized with ARNt, and binds to the DNA on target genes (Cytochrome family—activated in response to xenobiotics) (Ikuta et al., 2009. Biochemical pharmacology. 77:588-596). AhR expression was assayed by immunostaining in the living epidermis of human skin explant as follows: AHR immunostaining was performed on paraffinized sections with a monoclonal antibody anti-AHR (ThermoScientific, ref. MA1-514, clone RPT1), diluted 1:100 in PBS-BSA 0.3%-Tween 20 0.05% during 1 h at room temperature with the system Vectastain RTU Universal VECTOR streptavidine/biotine and revealed with VIP (Vector laboratories, ref. SK4600), a violet substrate of peroxydase. The immunostaining was performed using an automated slide processing system (Autostainer, Dako) and assessed by microscopical observation. Concerned batches: all the batches

Immunostaining of HO-1 (Heme-Oxygenase-1)

Heme-Oxygenase is a phase II skin anti-oxidant enzyme which generates cell anti-oxidant molecules. HO-1 expression is considered as a specific molecular indicator of cellular oxidative stressHeme oxygenase (HO) is a ubiquitous redox-regulated enzyme that degrades heme into biliverdin, which is rapidly converted to bilirubin by biliverdin reductase, carbon monoxide, and iron (Kutty et al, 1995. PNAS. 92:1177-1181; Maines, 1997. Ann Rev Pharmaco/Toxico/37:517-554). In mammals, HO-1 mRNA expression has been reported in various cells and tissues, including the skin (Hanselmann et al, 2001. Biochem J 353:459-466). Its expression is considered the most sensitive and reliable indicator of cellular oxidative stress (Kutty et al, 1995. PNAS. 92:1177-1181; Maines, 1997. Ann Rev Pharmacol Toxicol 37:517-554). It has been shown that UVA irradiation and other oxidants upregulate the expression of HO-1 mRNA in the skin (Keyse and Tyrrell, 1989; Keyse et al, 1990) and that the expression of HO-1 mRNA is induced by nitric oxide in cultured human keratinocytes (Clark et al, 1997). HO-1 expression was assayed by immunostaining in the living epidermis of human skin explant as follows: HO-1 immunostaining was performed on paraffinized sections with a monoclonal antibody anti-HO-1 antibody (Novus Biologicals, ref NBP1-97507, clone HO-1-1), diluted 1:25 in PBS-BSA 0.3%-Tween 20 0.05% over night at room temperature with the system Vectastain RTU Universal VECTOR streptavidine/biotine and revealed with VIP (Vector laboratories, ref. SK4600), a violet substrate of peroxydase. The immunostaining was performed using an automated slide processing system (Autostainer, Dako) and assessed by microscopical observation. Concerned batches: all the batches

Immunostaining of MT-1H

Metallothionein (MT) is a unique heat-stable protein with a metal binding domain consisting of 20 cysteine residues. MTF-1 (Metal Transcription Factor) is a Zn-responsive protein that regulates the transcription of a battery of genes involved in the defense against oxidative stress. More specifically, MTF-1 controls the transcription of metallothionein (MT) genes located on an area called MRE (Metal Response Element). Because of its high affinity to bind to and be induced by essential metals such as zinc and copper and harmful metals like cadmium and mercury, MT-1H is believed to play an important role in the homeostatic function of essential metals and in detoxification against toxic metals. MT-1H expression was assayed by immunostaining in the living epidermis of human skin explant as follows: MT-1H immunostaining was performed on paraffinized sections with a monoclonal antibody anti-MT-1H antibody (Dako, ref M0639, clone E9), diluted 1:200 in PBS-BSA 0.3%-Tween 20 0.05% 1 hour at room temperature with the system Vectastain RTU Universal VECTOR streptavidine/biotine and revealed with VIP (Vector laboratories, ref. SK4600), a violet substrate of peroxydase. The immunostaining was performed using an automated slide processing system (Autostainer, Dako) and assessed by microscopical observation. Concerned batches: all the batches.

### Image Analysis

The images analyses were performed on all the images (9 to 12 images) of each batch using the CellD storing software (Olympus Microscopy, Matrix Optics (M)SdnBhd, Taman Mayhang, Maylasia). For each batch of explants, the percentage of the region of interest covered by the staining (stained surface percentage) was determined by image analysis.

The stained surface percentage (Surf %) for each treatment (P) was compared to the stained surface percentage of untreated condition (T=control untreated condition), P vs. T comparison. The same comparisons were made for the exposed batches with (PML=osmolyte treatment in presence of heavy metal) or without treatment (ML=heavy metal exposure without osmolyte treatment), PML vs ML comparison.

### Inductions Comparisons

To take into account the unexposed levels in the conditions exposed to the pollutants, the ΔML was calculated as described below.

For the untreated condition, the mean Surf % value of the unexposed condition (T) was subtracted to each Surf % value of the condition exposed to the pollutants (ML). This represents the variation of the Surf % induced by the pollutants exposure=ΔML. (Comparison between the condition under pollution exposure, versus the non polluted condition. ΔML=(pollution condition results)−(non-polluted condition results)).

The ΔML for each treatment P was calculated using the same method to obtain the ΔPML, i.e. the variation of the Surf % induced by the pollutants exposure when treated with P.

Thus, the mean ΔML can be compared to the mean ΔPML (ΔPML vs ΔML.) List of the analyzed batches

### Analysis of AhR Immunostaining

Analyzed batches: T, P1, P2, P3, P6, ML, P1ML, P2ML, P3ML and P6ML (10 batches, 3 explants/batch, so 30 explants) at day 5 (referred to as TJ5, P1J5, P2J5, P3J5, P6J5, MLJ5, P1MLJ5, P2MLJ5, P3MLJ5 and P6MLJ5). TJ5: non polluted non treated control at day 5 (D5); P1J5: product A 4% non-polluted at D5; P2J5: product A 2% non-polluted at D5; P3J5: product A 1% non-polluted at D5 P6J5: product B 0.3% non-polluted at D5; MLJ5: polluted non treated control batch at D5; P1MLJ5: product A 4%/polluted at D5; P2MLJ5: product A 2%/polluted at D5.; P3MLJ5: product A 1%/polluted at D5; P6MLJ5: product B 0.3%/polluted at D5.)

### Analysis of Nfr2 Immunostaining

Analyzed batches: T, P1, P2, P3, ML, P1ML, P2ML and P3ML (8 batches, 3 explants/batch, so 24 explants), at day 5 (referred to as TJ5, P1J5, P2J5, P3J5, MLJ5, P1MLJ5, P2MLJ5, and P3MLJ5).

### Analysis of HO-1 Immunostaining

Analyzed batches: T, P2, ML, P2ML (4 batches, 3 explants/batch, so 12 explants at day 5 referred to as TJ5, P2J5, MLJ5, P2MLJ5.

### Analysis of MT-1H Immunostaining

Analyzed batches: T, P2, ML, P2ML (4 batches, 3 explants/batch, so 12 explants) at day 5 referred to as: TJ5, P2J5, MLJ5, P2MLJ5.

Results are shown in Table 14.

TABLE 14
Comparisons of batches T and ML

| | | | | | | | | P2 vs P6 |
|---|---|---|---|---|---|---|---|---|
| | | | | | | | | P2ML vs |
| | | | | | | | | P6ML |
| | | Osmolyte Product A | | | Product B (L-Proline) | | | ΔP2ML |
| Variations vs T or | | P1 | P2 | P3 | P4 | P5 | P6 | vs |
| ML on day 5 | | (4%) | (2%) | (1%) | (1%) | (0.5%) | (0.3%) | ΔP6ML |
| MDA | PML vs | −9% ns | −7% ns | −1% ns | −10% ns | −14% # | −18%* | |
| | ML | | | | | | | |
| | ΔPML | −44% # | −18% (ns) | −8% (ns) | −34% # | −48%* | −62%* | |
| | vs ΔML | | | | | | | |
| AHR | P vs T | +97%** | +70%* | +40% ns | [symbol] | [symbol] | −63%* | +78% |
| | PML vs | −45%** | −60%** | −12%** | [symbol] [symbol] [symbol] [symbol] [symbol] | [symbol] [symbol] [symbol] [symbol] | −15%** | −113% |
| | ML | | | | | | | |
| | ΔPML | −156%** | −161%** | −53%** | | | +23%** | −302% |
| | vs ΔML | | | | | | | |
| Nrf2 | P vs T | +71%** | +41%* | +17% ns | [symbol] | [symbol] | | |
| | PML vs | +53%** | −10% ns | −19% ns | [symbol] [symbol] [symbol] [symbol] | [symbol] [symbol] | | |
| | ML | | | | | | | |
| | ΔPML | +24% ns | −89%* | −75%* | | | | |
| | vs ΔML | | | | | | | |
| HO-1 | P vs T | | +39%** | | [symbol] | | | |
| | PML vs | | −29%** | | [symbol] | | | |
| | ML | | [symbol] [symbol] | | | | | |
| | ΔPML | | −132%** | | | | | |
| | vs ΔML | | | | | | | |
| MT-1H | P vs T | | −26% ns | | [symbol] | | | |
| | PML vs | | −36%** | | [symbol] [symbol] [symbol] | | | |
| | ML | | [symbol] [symbol] [symbol] [symbol] | | | | | |
| | ΔPML | | −37%** | | | | | |
| | vs ΔML | | | | | | | |

The arrow [symbol] indicates a slight decrease, while [symbol] indicates a slight increase.
The arrows [symbol] [symbol] indicate a moderate decrease, while [symbol] [symbol] indicate a moderate increase.
The arrows [symbol] [symbol] [symbol] indicate a quite clear decrease, while [symbol] [symbol] [symbol] indicate a quite clear increase.
The arrows [symbol] [symbol] [symbol] [symbol] indicates a clear decrease, while [symbol] [symbol] [symbol] [symbol] indicate a clear increase.
The arrows [symbol] [symbol] [symbol] [symbol] [symbol] indicates a very clear decrease, while [symbol] [symbol] [symbol] [symbol] [symbol] indicates a very clear increase.
The arrows [symbol] [symbol] [symbol] [symbol] [symbol] [symbol] indicates a strong decrease, while [symbol] [symbol] [symbol] [symbol] [symbol] [symbol] indicates a strong increase.
The arrow [symbol] indicates no variation.
ns = non-significant
# = significant with p < 0.1 (90%),
*= significant with p < 0.05 (95%),
**= significant with p < 0.01(99%)

### MDA Analysis

If we consider that without heavy metal ML, the products P1 to P6 induce a variation of MDA concentration, it is possible to compare the induction of MDA (with or without ML) for each explant and to compare the average of these diminutions to those of the blank batch.

The average of the blank batch TJ5 was calculated (154.0 nmol/L). Then, for each explant of MLJ5, this average value was subtracted from TJ5. That corresponds to calculating the delta (increase) of MDA induced by ML for each explant compared to the average of the batch without ML, it is ΔML.

The average delta of the MDA induction was calculated in presence of products P1 to P6 (ΔPML) using the same method.

These average deltas were then compared to the average delta of MDA induction without treatment (ΔML).

The exposure of skin explants to the mix of pollutants had a significant effect on lipid oxidation as the MDAΔHM (nmol/L) was 73.6 representing the control sample. Product A-P1 showed a MDAΔHM (nmol/L) of 40.9.

With regard to product A (After pollutant exposure) shown in Table 14, the product P1 significantly reduced the increase of MDA by 44%. The product P2 non significantly reduced the increase of MDA by 18%. The product P3 non significantly reduced the increase of MDA by 8%. As such, the product A exhibits a dose dependent anti-oxydative effect. Furthermore, the product A significantly reduced the oxidative stress induced by the pollutants exposure. These data indicated that the products comprising a combination of proline, serine, betaine and inositol, when exposed to skin explants, were capable of helping protect skin against oxidative stress as evidenced by a reduced pollution induced lipids peroxidation.

With regard to product B (After pollutant exposure), the product P4 reduced the increase of MDA by 34%. The product P5 reduced the increase of MDA by 48% and the product P6 reduced the increase of MDA by 62%. As such the product B exhibits a significant inversed dose dependent anti-oxydative effect. The strongest effect was obtained with the concentration 0.3% and the weakest effect with the concentration 1%.

### Nrf2 Induction Analysis

Without Pollutants (without Heavy Metal Exposure, −HM).

Control treatment showed 6.4% stained surface, P3 showed a 7.5% stained surface, P2 showed a 9%* stained surface and P1 showed a 10.9*% stained surface on day 5.

As shown in Table 14, the comparison of effect of products application on Nrf2 expression to batch TJ5 is show in row P vs T, and indicates that the product P1 induced a significant increase of 71%**, product P2 induced a significant increase of 41%*, and product P3 induced a non-significant increase of 17%. This data indicates that product A exhibits a dose dependent stimulation effect on Nrf2 expression, suggesting that it increases the amount of defense enzymes, thereby helping the skin to be ready to make the pollutants unharmful.

After Pollutants Exposure (with Heavy Metal Exposure, +HM)

Control+HM treatment showed 10.5% stained surface, P3+HM showed a 8.5% stained surface, P2+HM showed a 9.4% stained surface and P1 showed a 16.0**% stained surface on day 5.

The Effect of products application on Nrf2 expression induced by the pollutants, compared to the batch untreated with products (ΔPML vs ΔML) are shown in Table 14: The product P1 non-significantly increased the Nrf2 induction by 24% ns

The product P2 significantly decreased the Nrf2 induction by 89%* indicating that an effective amount of the skin care composition of example 1 significantly limits the pollution induced Nrf2 increase by 89%. The product P3 significantly decreased the Nrf2 induction by 75%*

These data indicate that after pollutant exposure, an effective amount of the skin care composition inhibits the effect of pollutants on Nrf2 induction with a dose dependent effect and at an effective amount of the skin care composition at 2 wt % almost totally inhibits the effect of the pollutants on Nrf2 induction.

### AHR Analysis

Without Pollutants (without Heavy Metal Exposure, −HR)

Control treatment showed 41% stained surface, P3 showed a 58% stained surface, P2 showed a 70%* stained surface and P1 showed an 82%** stained surface on day 5.

Table 14 shows On D5, the effect of products application on AhR expression, compared to the batch TJ5. As shown in Table 14, the product P1 (A at 4%) induced a significant increase of AhR expression by 97%. The product P2 (A at 2%) induced a significant increase of AhR expression by 70%. The product P3 (A at 1%) induced a non-significant increase of AhR expression by 40%. The product P6 (B at 0.3%) induced a significant decrease of AhR expression by 63%. P2 vs P6: the AHR expression was significantly higher by 78%**.

This indicates that product A exhibited a dose dependent stimulation effect on AHR expression, suggesting that it increases the amount of receptors (such as phase I detoxification enzymes like CYP450) helping the skin to be ready to fight against pollutants.

The product B reduced the AHR expression reducing the potential of the skin to resist to a pollutants exposure.

After Pollutants Exposure (with Heavy Metal Exposure, +HM)

The exposure of skin explants to heavy metals (control pollution exposure +HM) showed a significant effect on AhR induction as AhR was induced by 128%** versus the untreated Control (control−HM). Control+HM treatment showed 94.4% stained surface, P3+HM showed a 82.8**% stained surface, P2+HM showed a 37.7**% stained surface and P1 showed a 51.6**% stained surface on day 5.

Effect of products application on AHR expression induced by the pollutants, compared to the batch untreated with products (ΔPML vs ΔML): The product P1 (A at 4%) significantly decreased the AHR induction by 156%** The product P2 (A at 2% ) significantly decreased the AHR induction by 161%**. The product P3 (A at 1%) significantly decreased the AHR induction by 53%.

These data indicate that after pollutant exposure, an effective amount of the skin care composition almost totally inhibits the effects of the pollutants on AhR induction with a dose dependent effect.

The product P6 (B at 0.3%) significantly increased the AHR induction by 23%* .ΔP2ML vs ΔP6ML (Proline at 0.3% within the osmolyte combination vs Proline at 0.3% as such): the AHR induction was significantly lower by 302%**.

### HO-1 Conclusion

Without Pollutants (without Heavy Metal Exposure, −HM).

Control treatment showed 55.1% stained surface, P2 showed a 76.7*% stained surface on day 5.

As shown in Table 14, on day 5 (D5), effect of products application on HO-1 expression, compared to the batch TJ5 are as follows. The product P2 (A at 2%) induced a significant HO-1 increase of 39%**, suggesting that it increases the amount of anti-oxidants in cells, helping the skin to be ready to make pollutants less harmful or unharmful.

The product P4 (B at 1%) does not seem to affect the HO-1 expression.

After Pollutants Exposure (with Heavy Metal Exposure, +HM).

Control+HM treatment showed 91.5**% stained surface, and P2+HM showed a 65.0**% stained surface on day 5. The pollutants exposure of skin explants induced a significant increase of HO-1 expression by 66%** (=control pollution exposure+HM) versus the control untreated (control−HM) at day 5 (MLJ5 vs TJ5)

As shown in Table 14, the effect of products application on HO-1 expression, compared to the batch exposed to pollutants (MLJ5): The product P2 (A at 2%) induced a significant decrease of 29%**

As shown in Table 14, the effect of products application on HO-1 expression induced by the pollutants, compared to the batch untreated with products (ΔPML vs ΔML): The product P2 (A at 2%) significantly decreased the HO-1 induction by 132%**

These data indicate that, after pollutants exposure, the product A (P2) contributes to inhibiting the effect of the pollutants on HO-1 induction. The product B (P4) strongly reduced the effect of the pollutants, but less than product A.

### MT-1H Conclusion:

Without Pollutants (without Heavy Metal Exposure, −HM).

Control treatment showed 3.2% stained surface, P2 showed a 2.4**% stained surface on day 5.

After Pollutants Exposure (with Heavy Metal Exposure, +HM)

Control treatment +HM showed 44.7%** stained surface and P2 showed a 28.6%** stained surface on day 5.

The pollutants exposure of skin explants induced a significant increase of MT-1H expression by 1297%** (=control pollution exposure+HM) versus the control untreated (control−HM) at day 5 (MLJ5 vs TJ5)

As shown in Table 14, the effect of products application on MT-1H expression, compared to the batch exposed to pollutants (MLJ5): The product P2 (A at 2%) induced a significant decrease of about 36%**.

Effect of products application on MT-1H expression induced by the pollutants, compared to the batch untreated with products (ΔPML vs ΔML): The product P2 (A at 2%) significantly decreases the MT-1H induction by 37%**

After pollutants exposure, the product A (P2) almost totally inhibits the effect of the pollutants. The product B (P4) seems to strongly reduce the effect of the pollutants, but less than product A.

As shown in Table 14 (MT-1H data), The product A after pollutants exposure almost totally inhibited the effect of the pollutants. The product B after pollutants exposure strongly reduced the effect of the pollutants.

These data indicated that the products comprising a combination of proline, serine, betaine and inositol were capable of contributing to skin detoxification, as evidenced by decreased metal regulation marker MT-1H (Metallothioneine).

### Example 6

### Assessment of the Efficacy of Two Products Against UV-Induced Protein and DNA Damage on Human Living Skin Explant Ex Vivo

### Products

Same as Example 5.

### Explant Preparation

human skin explants of an average diameter of 12 mm (±1 mm) were prepared on an abdoplasty coming from a 41-year-old Caucasian woman). The explants were kept in survival in BEM culture medium at 37° C. in a humid, 5%-CO2 atmosphere.

The explants were distributed into 5 batches as shown in Table 15.

TABLE 15
Explant batches

| | | | Number of | Sampling |
|---|---|---|---|---|
| Batch | Treatment | Irradiation | explants | time |
| T0 | — | — | 3 | Day 0 |
| T | — | — | 3 | Day 4 |
| P1 | A at 4% | — | 3 | Day 4 |
| P2 | A at 2% | — | 3 | Day 4 |
| P3 | A at 1% | — | 3 | Day 4 |
| P4 | B at | — | 3 | Day 4 |
| | 10 mg/mL | | | |
| | (1%) | | | |
| P5 | B at 5 mg/mL | — | 3 | Day 4 |
| | (0.5%) | | | |
| P6 | B at 3 mg/mL | — | 3 | Day 4 |
| | (0.3%) | | | |
| TUV | UVA + UVB | 4 MED UVA + | 3 | Day 4 |
| | | 2 MED UVB | | |
| P1UV | A at 4% | 4 MED UVA + | 3 | Day 4 |
| | | 2 MED UVB | | |
| P2UV | A at 2% | 4 MED UVA + | 3 | Day 4 |
| | | 2 MED UVB | | |
| P3UV | A at 1% | 4 MED UVA + | 3 | Day 4 |
| | | 2 MED UVB | | |
| P4UV | Bat | 4 MED UVA + | 3 | Day 4 |
| | 10 mg/m L | 2 MED UVB | | |
| | (1%) | | | |
| P5UV | B at 5 mg/mL | 4 MED UVA + | 3 | Day 4 |
| | (0.5%) | 2 MED UVB | | |
| P6UV | B at 3 mg/mL | 4 MED UVA + | 3 | Day 4 |
| | (0.3%) | 2 MED UVB | | |

### Explant Treatment

On day 0 (D0), day 1, day 2 and day 3 (3 hours before irradiation), the tested products were applied topically on surface of skin explants at the rate of 2 mg/cm², twice a day for D1 and D2 and once on DO and D3. The blank batch T did not receive any treatment, except the renewal of the culture medium. The medium was half renewed (1 ml per well) on day 2.

### UV Irradiation

On D3, the culture media of all the batches was replaced by HBSS solution (Hank's Balanced Saline Solution; 1 ml per explant). The batches “UV” were irradiated by UVA+ UVB using a UV simulator Vibert Lourmat RMX 3W with a dose of 18 J/cm2 of UVA (4 MED, minimal erythemal dose) and 0.3 J/cm2 of UVB (2 MED). The unirradiated batches were kept in HBSS in the dark. At the end of the irradiation the explants were put back in 2 mL of BEM medium.

### Sampling

On Day 0, three explants from batch TO were collected and cut in two parts: one half was frozen at −80° C. and one half was fixed in buffered formol solution.

On Day 4, three explants from each batch were collected and processed in the same way than on DO.

### Histological Processing

After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The frozen samples were cut into 7-μm-thick sections using a Leica CM 3050 cryostat. Sections were then mounted on Superfrost® plus silanized glass slides. The microscopical observations were realized using a Leica DMLB or Olympus BX43 microscope. Pictures were digitized with a numeric DP72 Olympus camera with CellD storing software.

TUNEL Assay (DNA Damage Assay)

DNA oxidization is a type of damage that can be promoted by reactive oxygen species (ROS), generated by UV exposure. The TUNEL assay was conducted by immunostaining in the living epidermis of human skin explant as follows: DNA damage was assessed on paraffinized sections using a In Situ Cell Death Detection Kit (Roche, ref. 11 684 817 910) with the TUNEL reagent diluted at 1:2 in PBS for 1 hour at room temperature and the POD converter diluted at 1:4, then revealed by VIP (Vector, ref. SK-4600). The staining was assessed by microscopical observation. Batches analyzed were T, TUV, P1UV, P2UV, P3UV at day 4 also referred to as TJ4, TUVJ4, P1UVJ4, P2UVJ4, P3UVJ4 (T=Control batch non treated non irradiated; TJ4=T at Day 4 (D4); TUV: control batch non treated UV irradiated; TUVJ4=TJ4 at Day 4; P1J4: product A 4% non irradiated at D4; P2J4: product A 2% non irradiated at D4; P3J4: product A 1% non irradiated at D4; P1UV: product A 4% UV irradiated; P1UVJ4: product A 4% UV irradiated at D4 P2UV: product A 2% UV irradiated; P2UVJ4: product A 2% UV irradiated at D4; P3UV: product A 1% UV irradiated; P3UVJ4: product A 1% UV irradiated at D4

Immunostaining of Carbonylated Proteins (FTZ)

Protein carbonylation is a type of protein oxidation that can be promoted by reactive oxygen species (ROS), generated by UV exposure. FTZ marker assay was conducted by immunostaining in the living epidermis of human skin explant as follows: Carbonylated proteins were stained on frozen sections with the anti-carbonylated proteins probe (fluorescein-5-thiosemicarbazid-FTZ) (Lifetechnologies, Ref. F-121) diluted at 0.5 μM in MES-Na+ and incubated for 30 minutes in the dark and at room temperature. The staining was observed by epifluorescence and assessed by microscopical observations. Batches analyzed were T, TUV, P1UV, P2UV, P3UV at day 4 also referred to as TJ4, TUVJ4, P1UVJ4, P2UVJ4, P3UVJ4

TABLE 16
Cell Viability, DNA damage (TUNEL) and Carbonylated protein (FTZ)
analysis.

| | | DNA damage | Carbonylated |
|---|---|---|---|
| | Cell Viability | (TUNEL) | proteins (FTZ) |
| P1 vs TJ4 | ↔ | [symbol] | ↔ |
| P1 vs TUVJ4 | ↔ | ↔ | [symbol] |
| P2 vs TJ4 | ↔ | [symbol] | ↔ |
| P2 vs TUVJ4 | ↔ | [symbol] [symbol] [symbol] | [symbol] |
| P3 vs TJ4 | ↔ | [symbol] | [symbol] |
| P3 vs TUVJ4 | ↔ | [symbol] | [symbol] |
| P4 vs TJ4 | ↔ | [symbol] | ↔ |
| P4 vs TUVJ4 | ↔ | [symbol] [symbol] | [symbol] |
| P5 vs TJ4 | ↔ | ↔ | [symbol] |
| P5 vs TUVJ4 | ↔ | [symbol] [symbol] | [symbol] |
| P6 vs TJ4 | ↔ | [symbol] [symbol] [symbol] | [symbol] |
| P6 vs TUVJ4 | | [symbol] [symbol] [symbol] | [symbol] |

The arrow [symbol] indicates a slight decrease, while [symbol] indicates a slight increase.
The arrows [symbol] indicate a moderate decrease, while [symbol] [symbol] indicate a moderate increase.
The arrows [symbol] [symbol] indicate a quite clear decrease, while [symbol] [symbol] [symbol] indicate a quite clear increase.
The arrows [symbol] [symbol] indicates a clear decrease, while [symbol] [symbol] [symbol] [symbol] indicate a clear increase.
The arrows [symbol] [symbol] [symbol] indicates a very clear decrease, while [symbol] [symbol] [symbol] [symbol] [symbol] indicates a very clear increase.
The arrows [symbol] [symbol] [symbol] indicates a strong decrease, while [symbol] [symbol] [symbol] [symbol] [symbol] [symbol] indicates a strong increase.
The arrow ↔ indicates no variation.

As shown in Table 16, the product P4 had a good activity against the UV-induced DNA damage, as was evidenced by a strong inhibition of the increase of DNA damage (TUNEL assay) induced by UV irradiation. Product P4 also inhibited the formation of carbonylated proteins induced by UV irradiation.

The product P5 “L-Proline at 5 mg/ml” presents a good activity against the UV-induced DNA damage. Indeed, it totally inhibits the increase of DNA damage (TUNEL assay) induced by UV irradiation. Moreover, it slightly inhibits the formation of carbonylated proteins induced by UV irradiation.

The product P6 “L-Proline at 3 mg/ml” presents a good activity against the UV-induced DNA damage and a fairly good activity against UV-induced protein damage. Indeed, it totally inhibits the increase of DNA damage (TUNEL assay) induced by UV irradiation. Moreover, it slightly inhibits the formation of carbonylated proteins induced by UV irradiation.

TABLE 17A
DNA damage (TUNEL) and Carbonylated protein (FTZ) analysis of
Product A ( at three concentration P1, P2, P3). Raw data from image
analysis of TUNEL and TFZ markers.

| Marker | TUNEL assay (stained surface percentage) | | | | |
|---|---|---|---|---|---|
| Test | TJ4 | TUVJ4 | P1UVJ4 | P2UVJ4 | P3UVJ4 |
| condition | | | | | |
| Surface % | 25.0 | 39.5 | 32.3 | 16.7 | 24.2 |
| Mean | | | | | |
| Standard | 8.8 | 4.6 | 6.1 | 5.2 | 3.5 |
| deviation | | | | | |
| Marker | Carbonylated proteins (stained surface percentage) | | | | |
| Test | TJ4 | TUVJ4 | P1UVJ4 | P2UVJ4 | P3UVJ4 |
| condition | | | | | |
| Surface % | 33.3 | 80.6 | 57.3 | 65.4 | 58.2 |
| Mean | | | | | |
| Standard | 29.8 | 9.8 | 23.3 | 11.2 | 20.4 |
| deviation | | | | | |

TABLE 17B
DNA damage (TUNEL) and Carbonylated protein (FTZ) analysis.
Effect of UV irradiation on control batch untreated:

| | | Carbonylated proteins |
|---|---|---|
| TUV versus T | DNA Damage (TUNEL) | (FTZ) |
| TUVJ4 vs TJ4 | +58%** | +142%** |

TABLE 17C
DNA damage (TUNEL) and Carbonylated protein (FTZ) analysis. Effect
of product treatment on irradiated batches:

| | | DNA | |
|---|---|---|---|
| | | Damage | Carbonylated proteins |
| Product A vs TUV | | (TUNEL) | (FTZ) |
| P1UVJ4 (A at 4%) | vs TUVJ4 | −18%* | −29%* |
| P2UVJ4 (A at 2%) | vs TUVJ4 | −58%** | −19%** |
| P3UVJ4 (A at 1%) | vs TUVJ4 | −39%** | −28%* |

TUJV4 = the UV-irraciated control untreated batch at day 4 (TUVJ4)
# = significant with p <0.1 (90%),
* = significant with p <0.05 (95%),
** = significant with p <0.01(99%)

The control untreated non irradiated skin explant (TUNEL test) showed a 25% stained surface while the control untreated UV irradiated skin explant showed a 40% stained surface, indicating that the UV irradiation induced a significant increase of DNA damage formation by 58%** (TUVJ4 vs TJ4, Table 17B).

The Effect of products application on DNA damage formation (TUNEL assay), compared to the batch irradiated to UV (TUVJ4) is shown in Table 16 and indicated that product P1 induced a significant decrease of 18%*, product P2 induced a significant decrease of 58%**, and product P3 induced a significant decrease of 39%**.

These data indicate that Product A at 4% (P1), at 2% (P2) and at 1% (P3) present a good protection against the UV-induced DNA damage. Indeed, it significantly inhibited the increase of DNA damage (TUNEL assay) induced by UV irradiation. P2 totally inhibited the increase of DNA damage.

For the FTZ marker assay, the non-irradiated control untreated showed a 33.3% stained surface, the irradiated control untreated showed an 80.6%# stained surface, the irradiated+P2 treatment showed a 65.4%** stained surface and the irradiated+P1 treatment showed a 57.3%* stained surface, on day 4.

The UV irradiation induced a significant increase of carbonylated proteins formation by 142%** (TUVJ4 vs TJ4, Table 17B) on the control untreated_UV irradiated skin surface.

As shown in Table 16, the effect of products application on carbonylated proteins (FTZ assay), compared to the batch irradiated to UV (TUVJ4) indicated that product P1 induced a significant decrease in carbonylated protein induction of 29%, product P2 induced a significant decrease of 19% and product P3 induced a significant decrease of 28%.

These data indicate that Product A at 4% (P1), at 2% (P2) and at 1% (P3) significantly inhibited the formation of carbonylated proteins induced by UV irradiation.

### Example 7

### A Skin Care Lotion for Pollution Protection of the Skin

Provided herein are skin care lotions comprising an effective amount of a skin care composition described herein comprising a combination of the osmolytes L-Proline, L-Serine, Betaine and Inositol in a ratio of 30% L-Proline, 14% L-Serine, 50% Betaine, and 6% Inositol of total % osmolytes, respectively, for detoxification and pollution protection for the skin.

These lotions can provide a first layer of pollution protection for the skin with detoxification, moisturization and energizing benefits. Between a tonic and a serum, this “oil-in-essence” lotion features an innovative texture with oil droplets suspended in a lotion. In a drop of sensoriality, this emollient yet non greasy texture leaves the skin soft, supple and comfortable. For dehydrated skins that need an extra layer of comfort and protection before the serum. This lotion can be a translucent and colorless fluid gel with suspended oil droplets.

Shown in Table 18 is a formulation of a skin care lotion that is capable of providing pollution protection for the skin.

TABLE 18
Formulation of skin care lotion comprising an effective amount of the
skin care composition described herein containing the osmolytes
betaine, inositol, serine and proline

| PHASE | PRODUCT | INCI | % |
|---|---|---|---|
| A | Demineralized water | Aqua | 68.35 |
| | Swiss Thermal Spring | Aqua, Sodium Benzoate | 5.00 |
| | Water (P00020542¹) | | |
| | Glycerine 86.5% USP | Glycerin, Aqua | 10.00 |
| | (Centonze)² | | |
| | Hydrolite-5 616751³ | Pentylene Glycol | 5.50 |
| | Skin care composition | Aqua, Betaine, Proline, | 3.00 |
| | described | Serine, Inositol, Citric Acid, | |
| | herein (Example 1)⁴ | Sodium Benzoate | |
| B | Alcasealan⁵ | Alcaligenes Polysaccharides | 0.10 |
| C | Miglyol 829⁶ | Caprylic/Capric/Succinic | 8.00 |
| | PF Verbier 201200867 - | Triglyceride | 0.05 |
| | SA⁷ | Parfum | |

Suppliers:
¹Botanica,
²Spiga,
³Safic Alcan,
⁴DuPont,
⁵Nagase,
⁶Sasol,
⁷Cosmo Fragrances

The key ingredient of this skin care product is an effective amount of the skin care composition described herein (Example 1) comprising a combination of the osmolytes L-Proline, L-Serine, Betaine and Inositol in a ratio of 30% L-Proline, 14% L-Serine, 50% Betaine, and 6% Inositol of total % osmolytes, respectively, for detoxification and pollution protection.

Furthermore, the osmolytes, glycols and Swiss thermal spring water help to refresh, hydrate and energize the skin.

The skin care product can be prepared by mixing the components of phase A under homogenizer until complete dispersion with Phase B and C. Phase B can be dispersed under homogenizer for about 10 minutes to obtain a homogeneous gel, and Phase C (the fragrance phase) can be pre-dispersed and slowly added to the mixture of Phase A.

### Example 8

### A Skin Care Jelly for Pollution Protection for the Skin

Provided herein are skin care jellies comprising an effective amount of a skin care composition described herein comprising a combination of the osmolytes L-Proline, L-Serine, Betaine and Inositol in a ratio of 30% L-Proline, 14% L-Serine, 50% Betaine, and 6% Inositol of total % osmolytes, respectively, for detoxification and pollution protection of the skin.

This skin care jelly can be provided to a skin after providing the skin care lotion described in Example 7. The skin care jelly described in Table 18 can provide a second layer of pollution protection for the skin with detoxification, moisturization and energizing benefits. Thanks to its quick-break sorbet texture, this jelly melts instantly into skin with an instant fresh moisture feel. This fresh sorbet leaves the skin smooth with a non-tacky after-feel. Very light, this texture is suitable for all skin types, even the most sensitive. This jelly can be a white translucent gel with frosted texture.

Shown in Table 19 is a formulation of a skin care jelly that is capable of providing detoxification and pollution protection of the skin.

TABLE 19
Formulation of skin care jelly comprising an effective amount of the skin
care composition described herein containing the osmolytes betaine,
inositol, serine and proline.

| PHASE | PRODUCT | INCI | % |
|---|---|---|---|
| A | Dem ineralized water | Aqua | 76.05 |
| | Swiss Thermal Spring | Aqua, Sodium Benzoate | 5.00 |
| | Water P-00020542 ¹ | | |
| | Zemea ® Propanediol ² | Propanediol | 5.00 |
| | Hydrolite-5 616751 ⁴ | Pentylene Glycol | 6.00 |
| | | | 3.00 |
| | Skin care composition | Aqua, Betaine, Proline, Serine, | |
| | described | Inositol, Citric Acid, Sodium | |
| | herein (Example 1)³ | Benzoate | |
| B | Sodium Hydroxide 10%⁵ | Aqua, Sodium Hydroxide | 0.20 |
| C | Sensanov WR⁶ | C20-22 Alkyl Phosphate, C20- | 0.50 |
| | | 22 Alcohols | |
| D | Sepimax Zen⁶ | Polyacrylate Crosspolymer-6 | 0.20 |
| | Covacryl MV 60⁷ | Sodium Polyacrylate | 1.00 |
| E | DC 2501 Cosmetic Wax² | Bis-Peg-18 Methyl Ether | 3.00 |
| | | Dimethyl Silane | |
| F | PF Verbier 201200867 - | Parfum | 0.05 |
| | SA⁸ | | |

Suppliers:
¹ Botanica,
² Biesterfield,
⁴ Safic Alcan,
³DuPont,
⁵Naturalalps,
⁶Seppic,
⁷Mimox,
⁸Cosmo Fragrances

The key ingredient of this skin care product is an effective amount of the skin care composition described herein (Example 1) comprising a combination of the osmolytes L-Proline, L-Serine, Betaine and Inositol in a ratio of 30% L-Proline, 14% L-Serine, 50% Betaine, and 6% Inositol of total % osmolytes, respectively, for detoxification and pollution protection.

Furthermore, the osmolytes, Zemea® propanediol and Swiss thermal spring water help to refresh, hydrate and energize the skin.

The skin care jelly can be prepared as follows: Heat the water to 80° C., then mix A under homogenizer. 2. Neutralize under homogenizer. 3. Disperse B under homogenizer for 10 minutes. 4. Disperse D under homogenizer for 10 minutes. 5. Add E under homogenizer o for 5 minutes. 6. Add F under homogenizer.

### Example 9

### A Skin Care Cream for Pollution Protection of the Skin

Provided herein is a skin care cream comprising an effective amount of a skin care composition described herein comprising a combination of the osmolytes L-Proline, L-Serine, Betaine and Inositol in a ratio of 30% L-Proline, 14% L-Serine, 50% Betaine, and 6% Inositol of total % osmolytes, respectively, for skin detoxification, radiance, moisturization and pollution protection of the skin.

Pearlescent white soft beads of moisturizing cream, immersed in a detox pollution protection concentrate. After been broken, the pearls melt and blend with the serum into a luxurious cream, leaving a soft and velvety film, with no residue, for an instant skin radiance.

This skin care cream can be provided as white pearlescent soft beads immersed in a clear liquid.

Shown in Table 20 is a formulation of a skin care cream that is capable of providing detoxification and pollution protection for the skin.

TABLE 20
Formulation of skin care cream comprising an effective amount of the
skin care composition described herein containing the osmolytes betaine, inositol,
serine and proline.

| PHASE | PRODUCT | INCI | % |
|---|---|---|---|
| A | Demineralized water | Aqua | 5.55 |
| | Swiss Thermal Spring Water | Aqua, Sodium Benzoate | 5.00 |
| | P-00020542¹ | | |
| | Hydrolite-5 616751³ | Pentylene Glycol | 6.00 |
| | Skin care composition | Aqua, Betaine, Proline, Serine, | 3.00 |
| | described herein (Example 1)² | Inositol, Citric Acid, Sodium Benzoate | |
| B | Pf Flocons G111 32510 - SA⁴ | Parfum | 0.15 |
| | Cremophor RH 40 ⁵ | PEG-40 Hydrogenated Castor Oil | 0.30 |
| C | Starpearls Blanches SP- | Aqua, Glycerin, Butylene Glycol, | 80.0 |
| | RA5S-5V7080⁶ | Dipropylene Glycol, Peg/Ppg-17/6 | |
| | | Copolymer, Beta-Glucan, Propanediol, | |
| | | Bis-Peg-18 Methyl Ether Dimethyl | |
| | | Silane, Synthetic Fluorphlogopite, | |
| | | Panthenol, 1,2-Hexanediol, Agar, | |
| | | Hydroxyethyl Acrylate/Sodium | |
| | | Acryloyldimethyl Taurate Copolymer, | |
| | | CI 77891, PEG-60 Hydrogenated | |
| | | Castor Oil, Glyceryl Acrylate/Acrylic | |
| | | Acid Copolymer, Sodium Polyacrylate, | |
| | | Ethylhexylglycerin, Disodium Edta. | |

Suppliers:
¹Botanica,
²DuPont,
³Safic Alcan,
⁴Robertet,
⁵ BASF,
⁶GeorgesWalther

The key ingredient of this skin care product is an effective amount of the skin care composition described herein (Example 1) comprising a combination of the osmolytes L-Proline, L-Serine, Betaine and Inositol in a ratio of 30% L-Proline, 14% L-Serine, 50% Betaine, and 6% Inositol of total % osmolytes, respectively, for detoxification and pollution protection.

Furthermore, the osmolytes, glycols and Swiss thermal spring water help to refresh, hydrate and energize the skin, while pearlescent pigments help providing the instant radiance.

The skin care product can be prepared as follows: Mix A (at room temperature) under pale (helix) stirrer until complete dissolution. 2. Pre-disperse the fragrance. 3. Add C manually.

### Example 10

### A Skin Care Gel for Pollution Protection of the Skin

Provided herein is a skin care gel comprising an effective amount of a skin care composition described herein comprising a combination of the osmolytes L-Proline, L-Serine, Betaine and Inositol in a ratio of 30% L-Proline, 14% L-Serine, 50% Betaine, and 6% Inositol of total % osmolytes, respectively, for pollution protection of the skin, moisturizing and energizing the skin to help detoxifying the skin.

This memory shape solid-like gel texture can break like ice upon mixing and can be a colored solid-like clear gel. This skin care gel can be provided as a face gel.

Shown in Table 21 is a formulation of a skin care gel that is capable of providing detoxification and pollution protection of the skin.

TABLE 21
Formulation of skin care gel comprising an effective amount of the skin
care composition described herein containing the osmolytes betaine,
inositol, serine and proline.

| PHASE | PRODUCT | INCI | % |
|---|---|---|---|
| A | Osmosed water¹ | Aqua | 86.41 |
| B | Edeta BD² | Disodium Edta | 0.10 |
| C | Rheostyl 90N³ | Aqua, Acrylates/Beheneth- | 5.00 |
| | | 25 Methacrylate Copolymer | |
| D | Aqueous solution | Aqua, Sodium Hydroxide | 1.67 |
| | NaOH 10%⁴ | | |
| E | Zemea ® ⁴ | Propanediol | 1.00 |
| F | Minasolve Green A⁵ | Pentylene Glycol, | 2.50 |
| | | Phenylpropanol | |
| G | Skin care composition | Aqua, Betaine, Proline, | 1.00 |
| | described herein | Serine, Inositol, Citric Acid, | |
| | (Example 1)⁶ | Sodium Benzoate | |
| H | Lipobelle Glacier⁷ | Aqua, Alcohol, Lecithin | 2.00 |
| I | Arctic Sun Cloudberry | Glycerin, Aqua, Rubus | 0.01 |
| | Seed Extract⁹ | Chamaemorus Seed Extract | |
| J | Parfum S0009558 | Parfum | 0.10 |
| | Vegetal⁹ | | |
| K | Solution Colorante Blue | Aqua, CI 42090 | 0.10 |
| | 05601-J À 0.1% | | |
| L | Solution Colorante Red | Aqua, CI 17200 | 0.06 |
| | K7057-J À 0.1% | | |

Suppliers:
¹Effervescence Lab,
²Ami Chimie
³Coatex,
⁴Merck Chimie,
⁵IMCD,
⁶Verfilco,
⁷DuPont,
⁸Mibelle Biochemistry
⁹nter′ Actifs,
¹⁰Sozio.

The key ingredient of this skin care product is an effective amount of the skin care composition described herein (Example 1) comprising a combination of the osmolytes L-Proline, L-Serine, Betaine and Inositol in a ratio of 30% L-Proline, 14% L-Serine, 50% Betaine, and 6% Inositol of total % osmolytes, respectively, for detoxification and pollution protection.

Furthermore, Osmolytes, Zemea® propanediol, Swiss glacier water and antioxidant-rich Arctic Cloudberry help to refresh, hydrate and energize the skin.

The skin care product can be prepared as follows: 1. Cold process. 2. Spread B into A, under moderate mixing with deflocculating stirrer, until complete dissolution. 3. Add one by one ingredients C to L, under moderate mixing with deflocculating stirrer. Make sure the homogenization is complete between each addition.

### Example 11

### A Skin Care Emulsion for Pollution Protection of the Skin

Provided herein is a skin care emulsion comprising an effective amount of a skin care composition described herein comprising a combination of the osmolytes L-Proline, L-Serine, Betaine and Inositol in a ratio of 30% L-Proline, 14% L-Serine, 50% Betaine, and 6% Inositol of total % osmolytes, respectively, for skin detoxification, moisturization and pollution protection of the skin.

Upon application, the skin care emulsion can release water droplets gliding on the skin surface providing fresh morning dew sensations.

This skin care emulsion can be provided as a white solid cream.

Shown in Table 22 is a formulation of a skin care emulsion that is capable of providing detoxification and pollution protection of the skin.

TABLE 22
Formulation of skin care emulsion comprising an effective amount of the
skin care composition described herein containing the osmolytes betaine, inositol,
serine and proline.

| PHASE | PRODUCT | INCI | % |
|---|---|---|---|
| A | Waterdropsil¹ | Cyclopentasiloxane, Dimethicone, | 4.00 |
| | | Dimethicone/Vinyl Dimethicone | |
| | | Crosspolymer, PEG-10 Dimethicone. | |
| | Cetiol CC² | Dicaprylyl Carbonate, Tocopherol | 2.00 |
| | Tegosoft SH ³ | Stearyl Heptanoate, Stearyl Caprylate | 3.50 |
| | Kahlwax 1899 ⁴ | Ozokerite | 3.00 |
| | Xiameter Pmx-200 Silicone | Dimethicone | 5.00 |
| | Fluid 5CS⁵ | | |
| | Parfum SO009558 Vegetal⁶ | Parfum | 0.30 |
| B | Osmosed water⁷ | Aqua | 70.577 |
| | Sodium Chloride Emprove ⁸ | Sodium Chloride | 0.50 |
| | Glycerine 99.5% ² | Glycerin | 3.00 |
| | Zemea ® ⁹ | Propanediol | 2.00 |
| | Microcare Sb ¹⁰ | Aqua, Sodium Benzoate, Potassium | 1.00 |
| | | Sorbate | |
| | Aqueous solution Citric acid | Aqua, Citric Acid | 0.113 |
| | 10% ¹¹ | | |
| | Skin care composition | Aqua, Betaine, Proline, Serine, Inositol, | 3.00 |
| | described herein (Example 1)¹² | Citric Acid, Sodium Benzoate | |
| | Lipobelle Glacier ¹³ | Aqua, Alcohol, Lecithin | 2.00 |
| | Arctic Sun Cloudberry Seed | Glycerin, Aqua, Rubus Chamaemorus | 0.01 |
| | Extract ¹⁴ | Seed Extract | |

Suppliers:
¹Impag,
²Ami Chimie,
³ Adara France,
⁴ Brenntag,
⁵Univar,
⁶Sozio,
⁷Effervescence Lab,
⁸ Merck,
⁹ IMCD,
¹⁰ Thor,
¹¹ Merck Chimie,
¹²DuPont,
¹³ Mibelle Biochemistry,
¹⁴ Inter′ Actifs.

The key ingredient of this skin care product is an effective amount of the skin care composition described herein (Example 1) comprising a combination of the osmolytes L-Proline, L-Serine, Betaine and Inositol in a ratio of 30% L-Proline, 14% L-Serine, 50% Betaine, and 6% Inositol of total % osmolytes, respectively, for detoxification and pollution protection.

Furthermore, Osmolytes, Zemea® propanediol, Swiss glacier water and antioxidant-rich arctic Cloudberry help to refresh, hydrate and energize the skin.

The skin care product can be prepared as follows: 1. Mix all ingredients of phase A, except A5 and A6 and heat up to 80° C. 2. Mix all ingredients of phase B and heat up to 80° C. 3. Add A5 and A6 in the rest of Phase A right before emulsification 4. Proceed to emulsification: pour Phase B very slowly in Phase A while progressively increasing the mixing speed (1000 rpm max). 5. At 70-80° C., pour immediately the liquid emulsion into the packaging.

### Example 12

### A Skin Care Stick for Pollution Protection of the Skin

Provided herein is a skin care stick comprising an effective amount of a skin care composition described herein comprising a combination of the osmolytes L-Proline, L-Serine, Betaine and Inositol in a ratio of 30% L-Proline, 14% L-Serine, 50% Betaine, and 6% Inositol of total % osmolytes, respectively, for skin pollution protection of the skin, such as but not limiting to eye contour.

This skin care stick can be a solid stick that can provide an instant ice-cold effect and can melt at skin contact for a surprising fresh effect. The stick can be applied on the delicate zone of the eye contour and energizes and moisturizes the look.

Shown in Table 23 is a formulation of a skin care stick that is capable of providing detoxification and pollution protection of the skin.

TABLE 23
Formulation of skin care stick comprising an effective amount of the skin
care composition described herein containing the osmolytes betaine, inositol,
serine and proline.

| PHASE | PRODUCT | INCI | % |
|---|---|---|---|
| A | Demineralized water | Aqua | 44.26 |
| | Glycerine 86.5% USP (Centonze)¹ | Glycerin, Aqua | 44.00 |
| | Butylene Glycol (Centonze)² | Butylene Glycol | 2.50 |
| B | Sodium Stearate Op-100 V³ | Sodium Stearate | 3.60 |
| C | Spectrastat Phl⁴ | Propanediol, 1,2-Hexanediol, | 2.50 |
| | | Caprylhydroxamic Acid | |
| D | Skin care composition described | Aqua, Betaine, Proline, Serine, | 3.00 |
| | herein (Example 1)⁵ | Inositol, Citric Acid, Sodium | |
| | | Benzoate | |
| E | Cremophor RH 40 ⁶ | PEG-40 Hydrogenated Castor | 0.08 |
| | | Oil | |
| | PF Verbier 201200867 - SA⁷ | Parfum | 0.04 |
| F | Colorant Bleu N°1 Unicert 05601-J/ | Aqua, CI 42090 | 0.02 |
| | 0.01% H2O ⁸ | | |

Suppliers:
¹Spiga,
²Brenntag/Georges Walter,
³Erbsloeh,
⁴IMCD,
⁵DuPont,
⁶BASF,
⁷Cosmo Fragrances,
⁸ Mimox

The key ingredient of this skin care product is an effective amount of the skin care composition described herein (Example 1) comprising a combination of the osmolytes L-Proline, L-Serine, Betaine and Inositol in a ratio of 30% L-Proline, 14% L-Serine, 50% Betaine, and 6% Inositol of total % osmolytes, respectively, for detoxification and pollution protection.

Furthermore, osmolytes and glycols help to hydrate and energize the skin.

The skin care product can be prepared as follows: 1. Heat the water to 80° C. then mix A under pale (helix) stirrer. 2. Disperse B under pale (helix) stirrer for 10 minutes. 3. Add C under pale (helix) stirrer at 60° C. 4. Add D under pale (helix) stirrer at 60° C. 5. Pre-disperse the fragrance than add E into the mix under pale (helix) stirrer. 6. Adjust the color under pale (helix) stirrer. Hot pouring the stick between 50° C. et 60° C.

### Example 13

### A Skin Care Mask for Pollution Protection of the Skin

Provided herein is a skin care mask comprising an effective amount of a skin care composition described herein comprising a combination of the osmolytes L-Proline, L-Serine, Betaine and Inositol in a ratio of 30% L-Proline, 14% L-Serine, 50% Betaine, and 6% Inositol of total % osmolytes, respectively, for skin pollution protection of the skin, such as but not limiting to night pollution protection

This skin care mask can be a natural oil gel mask to be applied in the evening for skin detoxification, blue-light protection and moisturization. In contact with water, the gel mask will transform into a milky substance for easy rinse-off. It leaves the skin comfortable and clean, prepared for application of a night cream, such as the skin care cream described in Example 14.

The mask can be applied in the evening, by applying a thick layer of a clear viscous oil gel and leave it to pause for 15 min and rinsed-off with luke-warm water.

Shown in Table 24 is a formulation of a skin care mask that helps providing detoxification and pollution protection of the skin.

TABLE 24
Formulation of skin care mask comprising an effective amount of the skin
care composition described herein containing the osmolytes betaine, inositol,
serine and proline.

| PHASE | PRODUCT | INCI | % |
|---|---|---|---|
| A | Glycerin⁽¹⁾ | Glycerin | 32.9 |
| | DUB SE 15P⁽²⁾ | Sucrose Palmitate | 2.0 |
| | Aqua | Aqua (Water) | 3.0 |
| | Skin care composition described | Aqua, Betaine, Proline, Serine, | 2.0 |
| | herein (Example 1) ⁽³⁾ | Inositol | |
| B | DUB 810C⁽²⁾ | Coco-Caprylate/Caprate | 28.5 |
| | Refined Sunflower oil ⁽⁴⁾ | Helianthus Annuus Seed Oil | 27.96 |
| | Tocopherol Oil CLR⁽⁵⁾ | Glycine Soja (Soybean) | 0.5 |
| | | Oil, Tocopherol | |
| | Rose Hip⁽⁶⁾ | Rosa Canina Seed Oil | 2.0 |
| | Guaiazulene 100% Cryst⁽⁷⁾ | Guaiazulene | 0.04 |
| C | Parfum Neroli-Macadamia ⁽⁸⁾ | Fragrance | 1.0 |
| | Soliberine ® NAT⁽⁹⁾ | Aqua, Propanediol, Buddleja | 0.1 |
| | | Officinalis Flower Extract | |

Suppliers:
¹Cremer Oleo,
²Stearinerie Dubois,
³ DuPont Industrial Biosciences,
⁴ Sophim,
⁵CLR,
⁶ICSC,
⁷Symrise,
⁸ Technico Flor,
⁹Greentech.

The skin care product can be prepared as follows: 1. Prepare phase A by dispersing the sucro-ester into glycerin under moderate stirring. Heat up to 80° C. and add the other ingredients of Phase A.; 2. Heat the ingredients of Phase B at 80° C.; 3. Add Phase B into Phase A under high shear mixing. (Ultraturax/Silverson-5000 to 10 000 rpm); 4. Begin cooling. At T°<40° C., add the ingredients of Phase C.; 5. Pour into packaging when the formulation is still hot.

### Example 14

### A Skin Care Cream for Pollution Protection of the Skin

Provided herein is a skin care cream comprising an effective amount of a skin care composition described herein comprising a combination of the osmolytes L-Proline, L-Serine, Betaine and Inositol in a ratio of 30% L-Proline, 14% L-Serine, 50% Betaine, and 6% Inositol of total % osmolytes, respectively, for skin pollution protection of the skin, such as but not limiting to night pollution protection and blue light protection as well as skin detoxification.

This skin care cream can be a white or colored thick cream with a buttery-thick texture that melts upon application.

Shown in Table 25 is a formulation of a skin care cream that helps providing detoxification and pollution protection, such as blue light protection, of the skin.

TABLE 25
Formulation of skin care cream comprising an effective amount of the
skin care composition described herein containing the osmolytes betaine, inositol,
serine and proline.

| PHASE | PRODUCT | INCI | % |
|---|---|---|---|
| A | Osmosed water | Aqua | 45.9 |
| B | Satiaxane ™ CX 91⁽¹⁾ | Xanthan Gum | 0.2 |
| | Glycerine 99,5%⁽²⁾ | Glycerin | 5.0 |
| C | Zemea ® ⁽³⁾ | Propanediol | 5.0 |
| D | Emulium ® Mellifera⁽⁴⁾ | Polyglyceryl-6 Distearate, Jojoba | |
| | | Esters, Polyglycery1-3 Beeswax, | 6.0 |
| | | Cetyl Alcohol | |
| | Eumulgin ® SG⁽⁵⁾ | Sodium Stearoyl Glutamate | 0.5 |
| | Compritol ® CG 888⁽⁴⁾ | Glyceryl Behenate | 1.5 |
| | Lanette ® 16⁽⁵⁾ | Cetyl Alcohol | 2.0 |
| | Lipocire ™ A SG⁽⁴⁾ | C10-18 Triglycerides | 15.0 |
| | MOD Ref. 5181⁽⁴⁾ | Octyldodecyl myristate | 5.0 |
| | | Diheptyl Succinate, Capryloyl | |
| | Lexfeel ® N5⁽⁶⁾ | Glycerin/Sebacic Acid Copolymer | 5.0 |
| E | Nipaguard ® SCE⁽⁷⁾ | Sorbitan Caprylate, Propanediol, | 1.25 |
| | | Benzoic Acid | |
| F | Microcare ® NB⁽⁸⁾ | Sodium Benzoate | 0.15 |
| | Osmosed water | Aqua | 3.0 |
| G | Soliberine ® ref001169⁽⁹⁾ | Aqua, Propanediol, Buddleja | |
| | | Officinalis Flower Extract | 1.0 |
| H | Skin care composition described | Aqua, Betaine, Proline, Serine, | |
| | herein (Example 1) ⁽¹⁰⁾ | Inositol | 3.0 |
| I | Parfum Neroli-Macadamia⁽¹¹⁾ | Fragrance | 0.5 |

Suppliers:
¹Cargill,
²Ami Chimie ,
³DuPont Tate&Lyle,
⁴Gattefossé,
⁵BASF,
⁶Inolex Inc.,
⁷Clariant International Ltd.,
⁸Thor,
⁹Greentech,
¹⁰ DuPont Industrial Biosciences,
¹¹Technico Flor

The skin care formulation comprising the components of Table 25 can further comprise 1%, 2%, 3%, 4%, up to 5% of GENENCARE® OSMS BA. In one aspect the formulation is as shown in Table 26.

TABLE 26
Formulation of skin care cream comprising an effective amount of the
skin care composition described herein containing the osmolytes betaine, inositol,
serine and proline.

| PHASE | PRODUCT | INCI | % |
|---|---|---|---|
| A | Osmosed water | Aqua | 42.9 |
| B | Satiaxane ™ CX 91⁽¹⁾ | Xanthan Gum | 0.2 |
| | Glycerine 99,5%⁽²⁾ | Glycerin | 5.0 |
| C | Zemea ® ⁽³⁾ | Propanediol | 5.0 |
| D | | Polyglyceryl-6 Distearate, Jojoba | |
| | Emulium ® Melliferam⁽⁴⁾ | Esters, Polyglyceryl-3 Beeswax, | 6.0 |
| | | Cetyl Alcohol | |
| | Eumulgin ® SG⁽⁵⁾ | Sodium Stearoyl Glutamate | 0.5 |
| | Compritol ® CG 888⁽⁴⁾ | Glyceryl Behenate | 1.5 |
| | Lanette ® 16⁽⁵⁾ | Cetyl Alcohol | 2.0 |
| | Lipocire ™ A SG⁽⁴⁾ | C10-18 Triglycerides | 15.0 |
| | MOD Ref. 5181⁽⁴⁾ | Octyldodecyl myristate | 5.0 |
| | Lexfeel ® N5⁽⁶⁾ | Diheptyl Succinate, Capryloyl | |
| | | Glycerin/Sebacic Acid Copolymer | 5.0 |
| E | Nipaguard ® SCE⁽⁷⁾ | Sorbitan Caprylate, Propanediol, | 1.25 |
| | | Benzoic Acid | |
| F | Microcare ® NB⁽⁸⁾ | Sodium Benzoate | 0.15 |
| | Osmosed water | Aqua | 3.0 |
| G | Soliberine ® ref001169⁽⁹⁾ | Aqua, Propanediol, Buddleja | 1.0 |
| | | Officinalis Flower Extract | |
| H | Skin care composition described | Aqua, Betaine, Proline, Serine, | 3.0 |
| | herein (Example 1) ⁽¹⁰⁾ | Inositol | |
| I | Parfum Neroli-Macadamia⁽¹¹⁾ | Fragrance | 0.5 |
| J | GENENCARE ® OSMS BA⁽¹⁰⁾ | Betaine | 3.0 |

Suppliers:
¹Cargill,
²Ami Chimie ,
³DuPont Tate&Lyle,
⁴Gattefossé,
⁵BASF,
⁶Inolex Inc.,
⁷Clariant International Ltd.,
⁸Thor,
⁹Greentech,
¹⁰DuPont Industrial Biosciences,
¹¹Technico Flor

The skin care product shown in Tables 25-26 can be prepared as follows: 1. 1. Heat A to 75° C. in a main tank; 2. Premix B, add it to A and mix for 5 min at 75° C. until homogeneous; 3. Add Zemea® and keep mixing for 5 min at 75° C.; 4. Premix phase D at 75° C. for 5 min until homogeneous; 5. Proceed to emulsification by adding D into ABC at high speed mixing for 15 min at 75° C.; 6. Cool down slowly to 50° C., then high speed mixing for 2 min; 7. Let it cool down to 35° C. under slower mixing then add E to the main tank and mix 5 min; 8. Prepare F, by mixing for 5 min until homogeneous; and 9. Add F, then G, H, I and optionally J, one by one to the main tank. Mix for 5 min between each addition until homogeneous.

That what is claimed:

1. A skin care composition comprising a combination of at least four osmolytes selected from the group consisting of proline, serine, betaine and inositol, wherein said combination comprises 10%-20% proline, 5%-10% serine, 20-30% betaine and 1%-5% inositol on a weight basis relative to a total weight of said composition.

2. A skin care composition comprising a combination of at least four osmolytes selected from the group consisting of proline, serine, betaine and inositol, wherein said combination comprises 30% proline, 14% serine, 50% betaine and 6% inositol on a % osmolyte basis relative to a total osmolyte content of 100%.

3. The skin care composition of any one of claims 1-2, further comprising at least one additional compound selected from the group consisting of a preservative and a pH adjuster.

4. The skin care composition of claim 3, wherein the additional compound is a preservative.

5. The skin care composition of claim 4, wherein the preservative is sodium benzoate

6. The skin care composition of claim 3, wherein the additional compound is a pH adjuster.

7. The skin care composition of claim 6, wherein the pH adjuster is citric acid.

8. The skin care composition of any preceding claims, wherein said skin care composition is a clear aqueous solution.

9. Use of an effective amount of the skin care composition of any preceding claim in a skin care product.

10. A skin care product comprising an effective amount of the skin care composition of any one of claim 1-8 and one or more dermatologically or cosmetically acceptable component.

11. The skin care product of claim 10, wherein said effective amount of the skin care composition is at least about 1%, 2%, 3%, 4% up to 5% on a weight basis relative to a total weight of said skin care product.

12. The skin care product of claim 10, wherein the one or more dermatologically acceptable component is a dermatologically acceptable carrier comprising about 10% to about 99% on a weight basis relative to a total weight of said skin care product.

13. A skin care product comprising a combination of the osmolytes proline, serine, betaine and inositol in a ratio of 30:14:50:6.

14. The skin care product of claims 10-13, wherein said skin care product is selected from the group consisting of a lotion, a serum, a jelly, a cream, a gel, an emulsion, a mask, a patch, and a stick.

15. A method for providing an increased skin care benefit to a skin, said method comprising contacting a skin with a skin care product comprising an effective amount of the skin care composition of any one of claims 1-8 and one or more dermatologically or cosmetically acceptable components.

16. The method of claim 15 wherein said increased skin care benefit is selected from the group consisting of an increased osmoprotection, an increased UV irradiation protection, and a combination thereof.

17. The method of claim 15, wherein said skin care product is a lotion, a serum, a jelly, a cream, a gel, an emulsion, a mask, a patch, or a stick comprising at least 1%, 2%, 3%, 4% up to 5% of said skin care composition on a weight basis relative to a total weight of said skin care product.
